(19) United States
(12) Patent Application Publication
(10) Pub. No.: US 2022/0050119 A1
(43) Pub. Date: Feb. 17, 2022

(54) BIOMARKERS

(71) Applicant: ProteinLogic Limited, Cambridge (GB)

(72) Inventors: Alexander BETZ, Cambridge (GB); Adrian WOOLFSON, Cambridge (GB)

(73) Assignee: ProteinLogic Limited, Cambridge, EN (GB)

(21) Appl. No.: 17/310,054

(22) PCT Filed: Jan. 17, 2020
(86) PCT No.: PCT/GB2020/050094

§ 371 Date: Jul. 14, 2021

(30) Foreign Application Priority Data
Jan. 17, 2019 (GB) 1900677.4

(51) Int. Cl. G01N 33/68; G01N 33/569

(52) U.S. Cl. CPC G01N 33/6893; G01N 33/6866; G01N 33/6869; G01N 33/5695; G01N 2333/57; G01N 2333/5434; G01N 2333/7151; G01N 2333/70596; G01N 2333/521; G01N 2333/5421; G01N 2333/5428

(57) ABSTRACT

The invention relates to panels of biomarkers for diagnosing and/or monitoring the progression of an active mycobacterial infection or for diagnosing the absence of a mycobacterial infection, particularly tuberculosis. Such diagnosis and/or monitoring may be differential diagnosis between active tuberculosis patients and patients with latent, non-progressing tuberculosis or healthy or sick patients, irrespective of whether the patients have been characterised as being sputum smear positive or sputum smear negative, and/or irrespective of whether they have been characterised as being HIV positive or HIV negative. The above pertain in all aspects both to pulmonary and extra pulmonary Mycobacterium tuberculosis infections, with Mycobacterium tuberculosis being the causative organism in tuberculosis.

### FIELD OF THE INVENTION

The invention relates to panels of biomarkers for diagnosing and/or monitoring the progression of an active mycobacterial infection or for diagnosing the absence of a mycobacterial infection, particularly tuberculosis. Such diagnosis and/or monitoring may be differential diagnosis between active tuberculosis patients and patients with latent, non-progressing tuberculosis or healthy or sick patients, irrespective of whether the patients have been characterised as being sputum smear positive or sputum smear negative, and/or irrespective of whether they have been characterised as being HIV positive or HIV negative. The above pertain in all aspects both to pulmonary and extrapulmonary Mycobacterium tuberculosis infections, with Mycobacterium tuberculosis being the causative organism in tuberculosis.

### BACKGROUND OF THE INVENTION

Mycobacterium tuberculosis is arguably one of the most successful pathologic micro-organisms worldwide and is the causative agent of the potentially lethal infectious disease tuberculosis (TB). It is also the leading cause of death worldwide from a potentially curable infectious disease, with an estimated two million related deaths annually.

The pathogenesis of TB is complex, with the initial infection occurring as the result of the inhalation of aerosolized infectious Mycobacterium tuberculosis. The immunological response to the bacillary insult dictates whether the infected individual will proceed to develop either localized pulmonary disease, latent disease (LTBI) or disseminated disease as a consequence of haematogenous spread. Those acquiring latent disease remain asymptomatic but retain the potential to evolve/progress into active disease (this occurs in approximately 10% of all cases over a lifetime period).

It is estimated that approximately one third of the world's population is infected with either latent or active TB. Figures from 2017 report approximately 6000 cases of active TB in the UK. This represents an overall upward trend over the last decade. The number of Mycobacterium tuberculosis isolates resistant to antibiotics is also increasing.

Pulmonary TB is the most common clinical presentation of infection with Mycobacterium tuberculosis. Symptoms typically include chronic cough with or without haemoptysis, fever, night sweats, and weight loss. Only individuals with active pulmonary disease remain infectious as they have the ability to aerosolize bacilli. Infection of almost any other organ system may occur with diverse accompanying clinical presentations. Immunocompromised individuals may present atypically.

The pathogen has shown a dramatic resurgence, driven in part by the HIV epidemic in sub-Saharan Africa, as the immunological response of these individuals is compromised. This has a two-fold impact. Firstly, individuals are more likely to progress from pre-existing latent disease to active disease and secondly, primary infection is more likely to take the form of active disease with increased infectivity. With increasing globalisation, the detection, prevention and early appropriate treatment of this aerosolised pathogen is becoming an increasingly important public health priority.

Despite extensive research, the current understanding of the immunological response and pathogenesis of Mycobacterium tuberculosis remains incomplete. Furthermore, the existing diagnostics and treatment methods are suboptimal. TB is definitively diagnosed by the identification of the causative organism (Mycobacterium tuberculosis) in a clinical specimen. This is achieved by prolonged culture of the organism, or by PCR analysis. Adjuncts to definitive diagnosis include: diagnostic imaging (X-rays or radiological scans), tuberculin skin tests (Mantoux/Heaf tests) and Interferon Gamma Release Assays (IGRAs).

Existing barriers to rapid definitive TB diagnosis include the difficulty in culturing this slow-growing organism in the laboratory, which can take around 3 to 12 weeks, or in obtaining an appropriate sample containing Mycobacterial DNA for PCR. The latter may require invasive sampling in the case of extrapulmonary TB, which is costly and may involve additional risks to the patient.

Developments in the field of TB diagnostics include the Xpert MTB/RIF test which has been endorsed by the World Health Authority. This is for use in sputum samples to diagnose cases of suspected active pulmonary TB, and to detect rifampicin resistant mutations, which are a marker for multi-drug resistant TB. This methodology is, however, dependent on a PCR-positive sample being obtained.

As a consequence of suboptimal diagnostic tools, anti-microbial combination chemotherapy treatment is often commenced empirically on the basis of clinical suspicion in conjunction with the results of adjunctive diagnostic tests. The rationale for this approach is two-fold. The patient receives a therapeutic trial of anti-microbial chemotherapy, and in cases of pulmonary TB, transmission is curtailed through the reduction of the bacillary load in the sputum.

Current diagnostic adjuncts include: radiological imaging, IGRAs and tuberculin skin testing (TST). Imaging provides guidance as to whether typical features are present, whereas IGRAs and TST provide information regarding possible prior exposure to M. tuberculosis by interrogating the immunological response to TB-related antigens. Interpretation of both the IGRA and TST tests is complex and confounded by a number of factors. These include amongst several others: prior exposure (latent disease), prior vaccination with BCG or other vaccines, and immunosupression. IGRA has, however, increasingly become an accepted adjunctive tool in countries with a low prevalence of TB for evaluating the likelihood of tuberculous infection being present. Unfortunately, imaging, TST and the IGRA test are unable to definitively diagnose or exclude the presence of active disease and no test currently available can identify or exclude the presence of extrapulmonary TB.

Publications have emphasized the potential for utilising combinations of biomarkers as diagnostic tools for TB. WO 03/075016 describes that levels of soluble proteins detectable in the blood, namely soluble CD antigens (“sCD”) sCD15, sCD23, sCD27 and sCD54, may be altered in patients with TB. A Medscape Medical News article from the American Thoracic Society (ATS) 2010 International Conference indicated that a combination of IL-15 and MCP-1 accurately categorised 84% of subjects as having active or latent TB. Chegou (2^{nd} Global Symposium on IGRAs May-June 2009) describes that combinations of biomarkers are more promising TB diagnostics than individual biomarkers and suggests measurement of EGF, sCD40L, MIP-1β, VEGF, TGF-α or IL-1α as a rapid test for active TB. Wu et al (2007) J Immunol 178, 3688-3694 indicated that expression of IL-8, FOXP3 and IL-12β mRNA offer a means of differentiating between latent M. tuberculosis infection and active TB disease.

As current diagnostic methodologies for TB are suboptimal, so too are the treatment options available for this disease. As a consequence of the intracellular nature of this pathogen, and lack of adequate innovations in the field of TB therapeutics, the basis of TB therapy continues to be combination anti-microbial chemotherapy over a prolonged period of months in the simplest cases. Partial compliance with treatment may lead to suboptimal therapeutic levels of the antimicrobials, and the micro-evolution of antibiotic resistance mutations. As a result of this, patients may remain infectious for longer durations, and the frequency of transmission is enhanced. The development of these resistance mutations, some of which are unresponsive to all known anti TB medications (multi-resistant), has recently caused heightened concern in a number of countries.

Furthermore, treatment options use invasive, potentially debilitating and costly therapeutics in patients which have been diagnosed using suboptimal diagnostic tools and therefore may not be infected with M. tuberculosis. Such treatment is unnecessary, reduces quality of life and may increase the likelihood of the development of antibiotic resistance mutations. Thus, there is a clear need for tests which are able to rule out active TB disease in patients who present with TB-like symptoms.

There is therefore a significant need to identify more effective and efficient methods for definitively ruling out active TB disease and for definitively diagnosing both active and latent TB, and in particular for differentiating active from latent TB and for diagnosing extrapulmonary TB.

### SUMMARY OF THE INVENTION

According to a first aspect of the invention, there is provided the use of a panel comprising the biomarkers IFN gamma, IL-10, CD120b and CD14 for diagnosing, and/or monitoring the progression of, an active mycobacterial infection.

According to a further aspect of the invention, there is provided a method of diagnosing the absence of an active mycobacterial infection in an individual, comprising:
(a) obtaining a test biological sample from an individual;
(b) quantifying the amount of the biomarkers as defined herein;
(c) inputting said amounts into a prediction model generated by an algorithm which comprises the relative amounts of each biomarker in active mycobacterial infected patients;
(d) obtaining a likelihood value for the absence of an active mycobacterial infection from said model.

According to a further aspect of the invention, there is provided a method of excluding the presence, and/or monitoring the progression of, an active mycobacterial infection in an individual thereto, comprising:
(a) obtaining a test biological sample from an individual;
(b) quantifying the amount of the biomarkers as defined herein;
(c) comparing the amounts of the biomarkers in the test biological sample with the amounts present in one or more control samples, such that a difference in the level of the biomarkers in the test biological sample is indicative of the absence of an active mycobacterial infection.

According to a further aspect of the invention, there is provided a method of determining the efficacy of anti-mycobacterial therapy for an active mycobacterial infection in an individual subject comprising:
(a) obtaining a biological sample from an individual;
(b) quantifying the amount of the biomarkers as defined herein;
(c) comparing the amounts of the biomarkers in the test biological sample with the amounts present in one or more control samples, such that a difference in the level of the biomarkers in the test biological sample is indicative of a response to the treatment.

According to a further aspect of the invention, there is provided a method of treating an active mycobacterial infection in an individual in need thereof, wherein said method comprises the following steps:
(a) excluding the presence, and/or monitoring the progression of, an active mycobacterial infection in an individual according to the method as defined herein; followed by
(b) administering an anti-mycobacterial medicament to said individual in the event of a positive diagnosis for active mycobacterial infection.

According to a further aspect of the invention, there is provided a kit comprising a biosensor capable of detecting and/or quantifying the amount of the biomarkers as defined herein, for use in diagnosing, excluding the presence of, and/or monitoring the progression of, an active mycobacterial infection.

### BRIEF DESCRIPTION OF THE FIGURES

FIG. 1: Biomarker expression in patient samples.

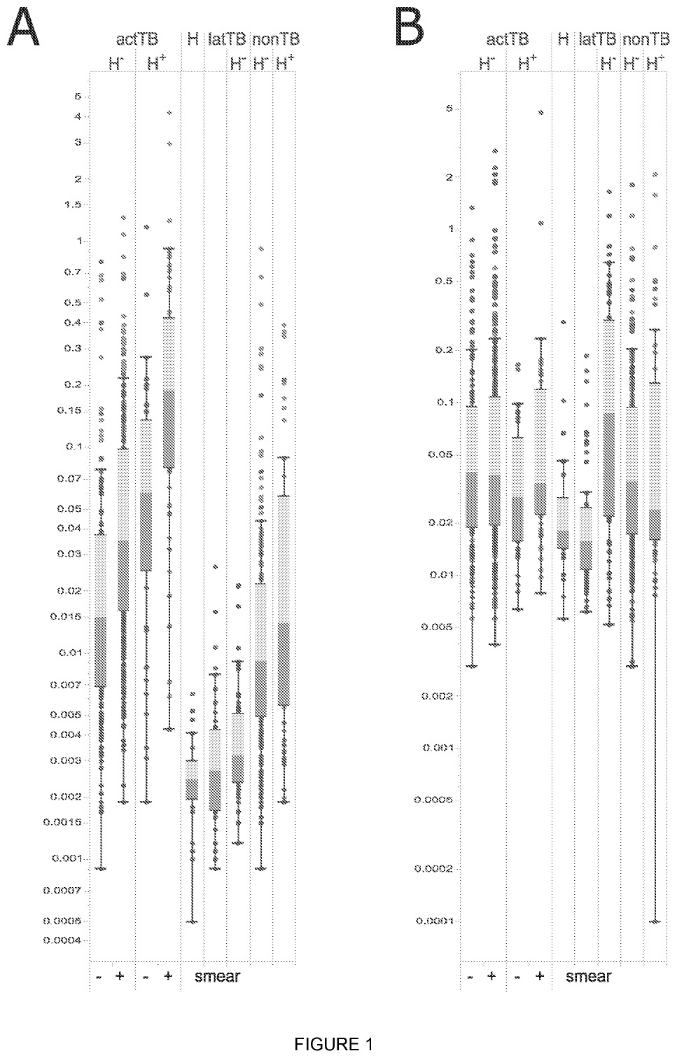

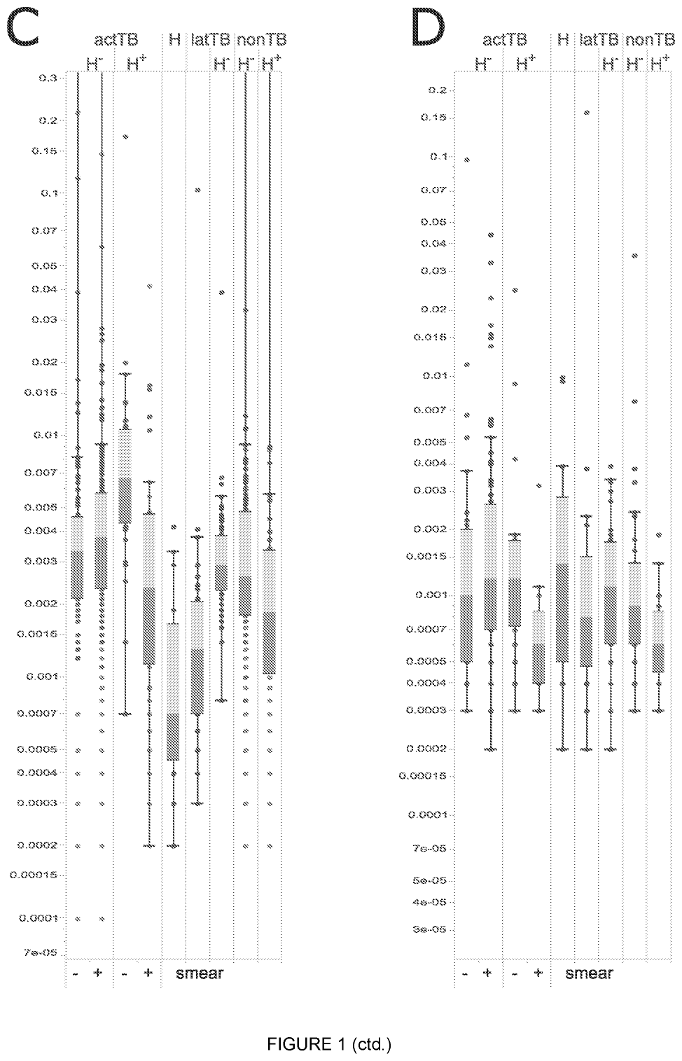

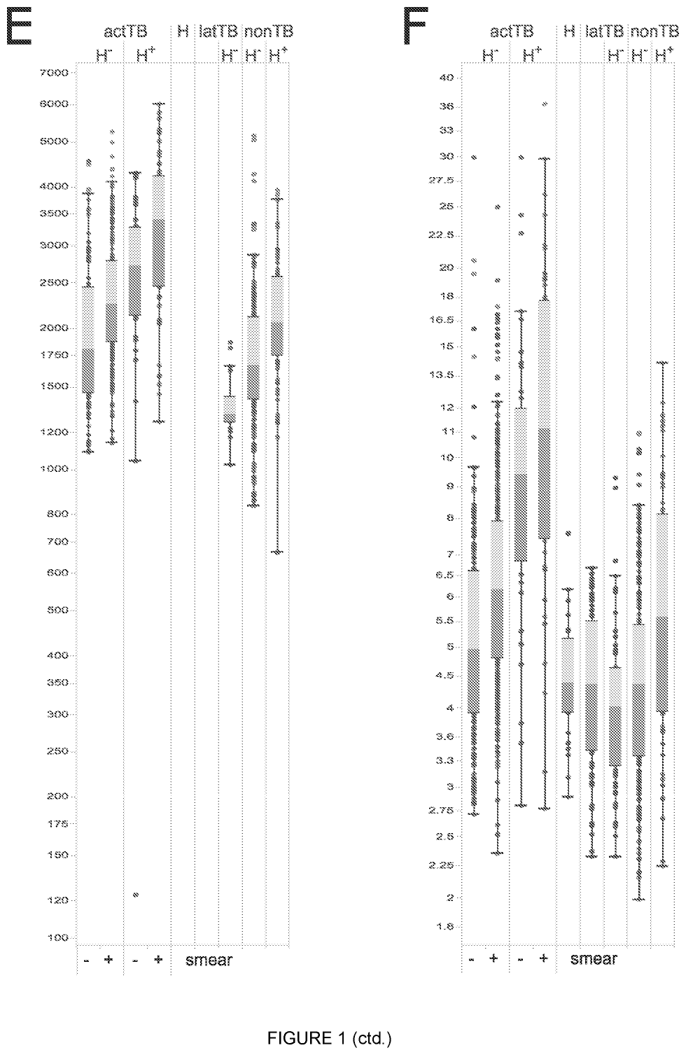

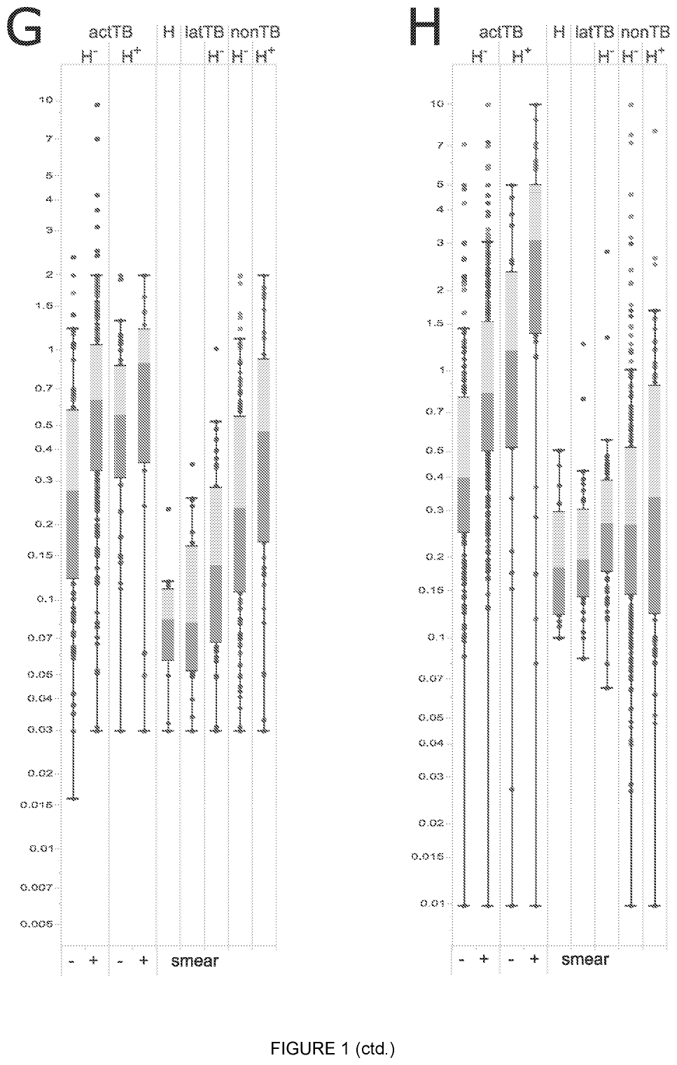

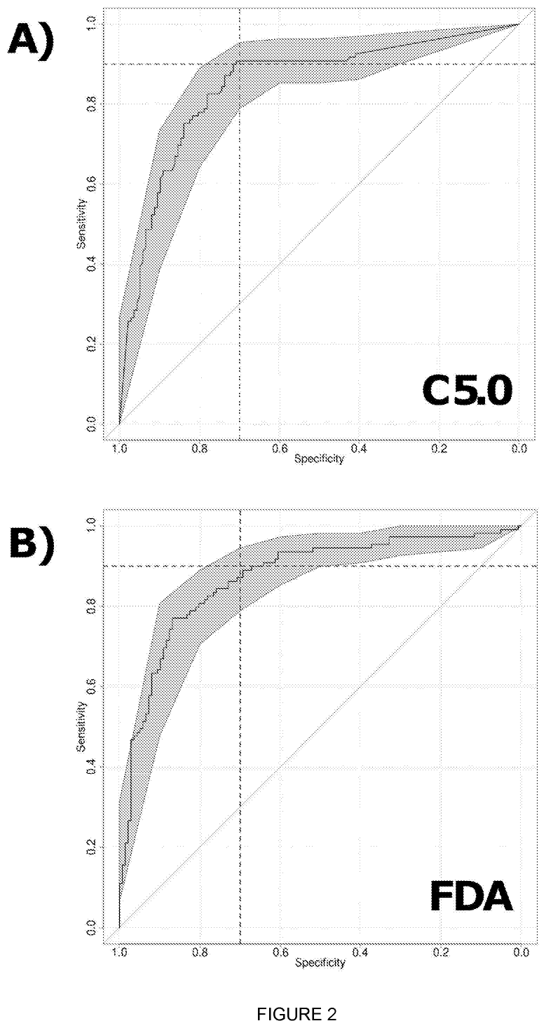

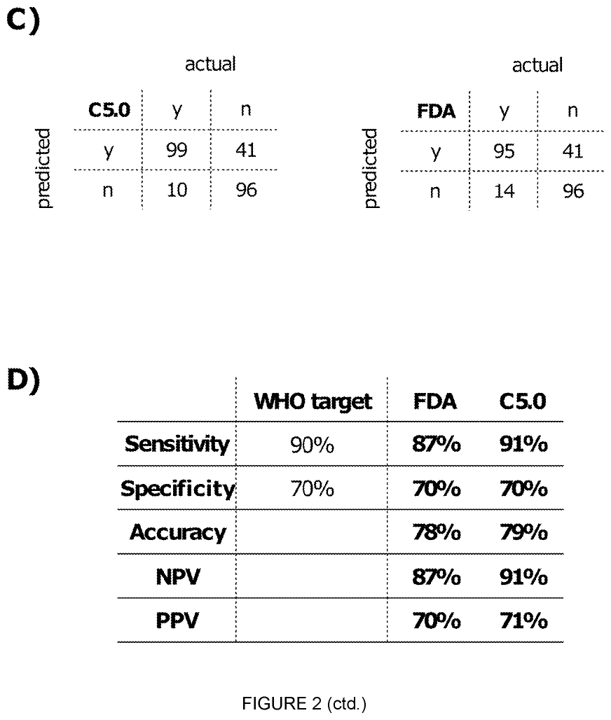

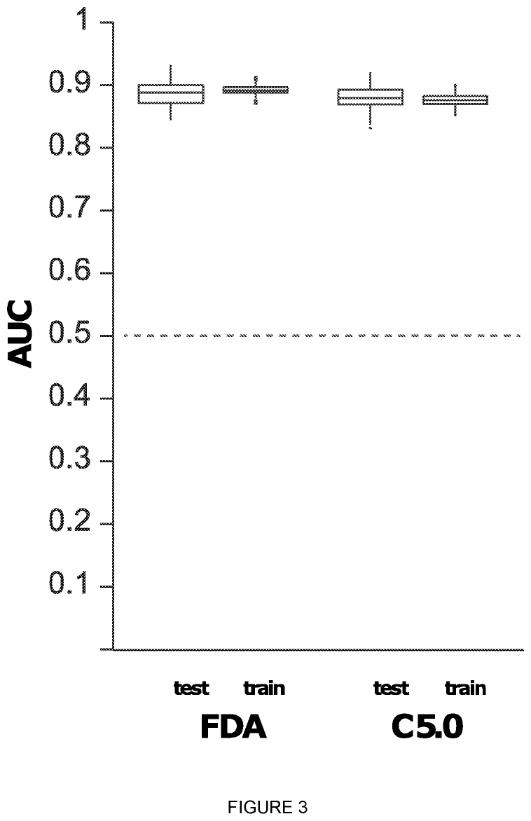

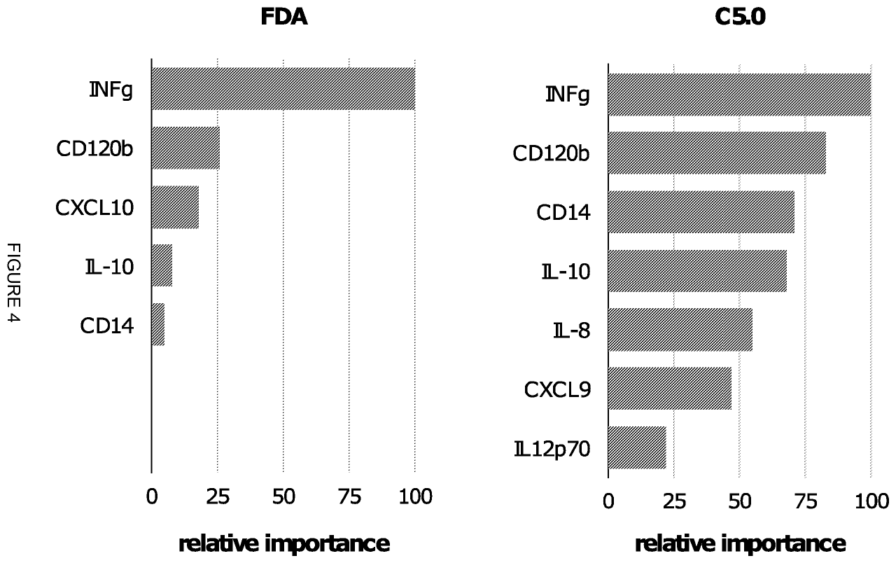

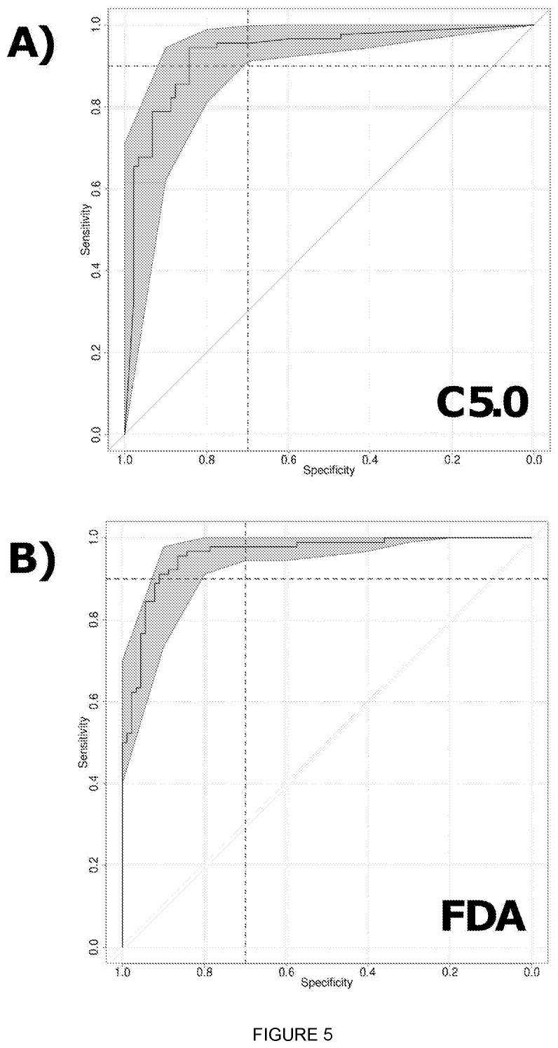

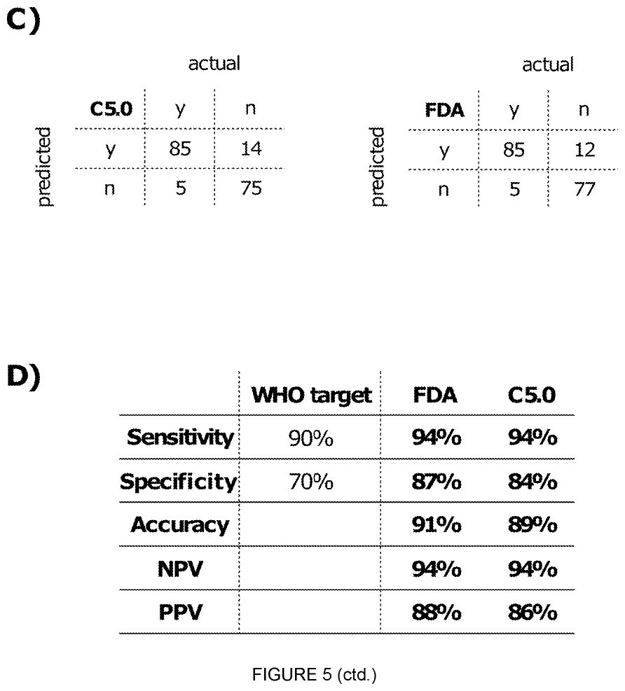

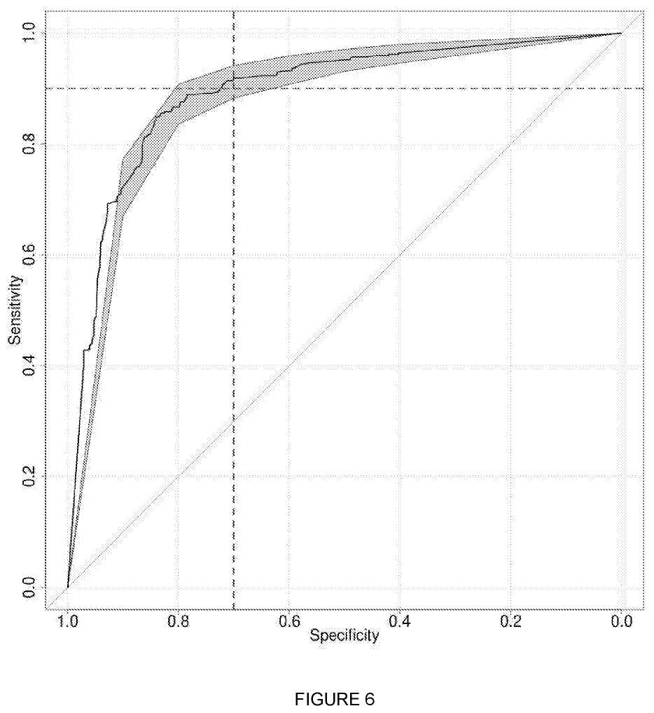

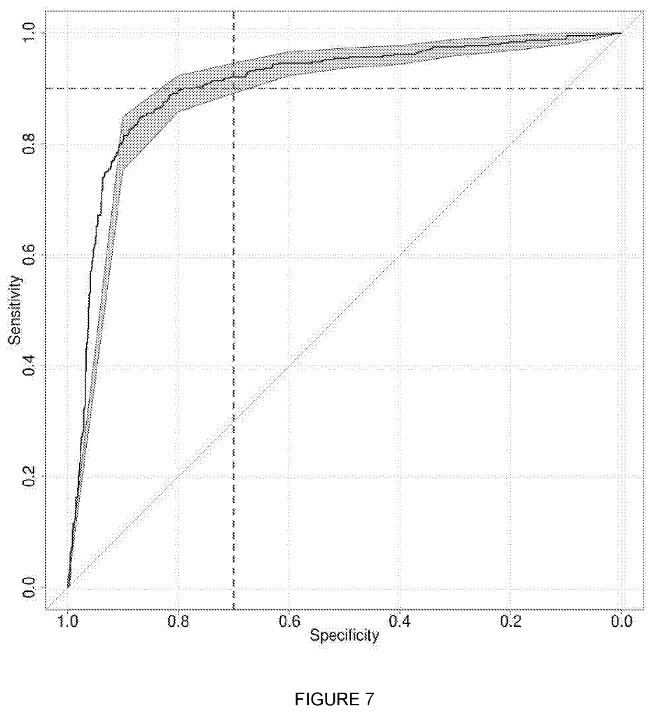

Boxplots of the expression level of A) IFNg (1181 samples), B) IL-8 (1180 samples), C) IL-10 (1181 samples), D) IL-12p70 (1181 samples), E) CD14 (599 samples), F) CD120b (1181 samples), G) CXCL9 (1165 samples) and H) CXCL10 (1181 samples). The samples were obtained from 5 independent patient cohorts. For those samples where the HIV status was known the results are for HIV positive (H+) and HIV negative (H−) patients are shown in separate columns. Analogously, results from smear positive (+) and smear negative (−) patients are shown in different columns. The concentration of the samples is shown in ng/ml on the y axis.

FIG. 2: Initial Assessment of the predictive models for ruling out active TB.

The predictive models were generated using a training dataset of 570 patient samples from the IDEA cohort (Table 2). A) Flexible Discriminant Analysis (FDA) and B) C5.0 classification model (C5.0) were used to generate the models. The FDA analysis uses the 5BM biomarker panel whereas the C5.0 model uses the 7BM biomarker panel (see FIG. 4). The Receiver Operator Characteristics (ROC) curves were computed using blindly chosen, independent test dataset of 246 patient samples from the IDEA cohort (Table 2). The dotted lines denote the high priority target product profile 2 (TPP2) set out for the WHO for a triage test (rule out) for active TB. The 95% confidence interval is shown as shaded. C) Confusion tables for the predicted versus actual diagnosis of active TB (γ) and all others (n) made by the C5.0 and FDA models. D) Summary of predictive model performance shown in A) and B) versus the WHO target product profile 2 (TPP2).

FIG. 3: In depth analysis of predictive models by re-assessment of random splits.

The original training and test data from the IDEA cohort (Table 2) were combined and resampled 100 times by randomly splitting them into training and test (70% and 30% of samples respectively, balanced for diagnosis, HIV status, and smear results). The distribution of area under the curve (AUC) values obtained at each iteration by applying the trained model to the training and testing data are shown. The results are plotted as box plots.

FIG. 4: Importance of biomarkers for the predictive models.

The mean relative variable importance for the biomarker predictors that are used by the FDA (5BM) and C5.0 (7BM) predictive models, across 100 random splits of the IDEA cohort into training and test data (70% and 30% of samples respectively, balanced for diagnosis, HIV status, and smear results).

FIG. 5: Assessment of predictive models using prospective patient data.

The predictive models were generated using a training dataset of 570 patient samples (see FIG. 2). A) Flexible Discriminant Analysis (FDA) and B) C5.0 classification model (C5.0) were used to generate the models. The Receiver Operator Characteristics (ROC) curves were computed using independent test dataset of 179 patient samples (see Table 5). The dotted lines denote the high priority target product profile 2 (TPP2) set out for the WHO for a triage test (rule out) for active TB. The 95% confidence interval is shown as shaded. C) Confusion tables for the predicted versus actual diagnosis of active TB (y) and all others (n) made by the C5.0 and FDA models D) Summary of predictive model performance shown in A) and B) versus the WHO target product profile 2 (TPP2).

FIG. 6: Receiver Operator Characteristics (ROC) curve for the 5BM biomarker panel as measured in the ELLA device as described in Example 3.

FIG. 7: Receiver Operator Characteristics (ROC) curve for the 7BM biomarker panel as measured in the ELLA device as described in Example 3.

### DETAILED DESCRIPTION OF THE INVENTION

According to a first aspect of the invention, there is provided the use of a panel comprising the biomarkers IFN gamma, IL-10, CD120b and CD14 for diagnosing and/or monitoring the progression of an active mycobacterial infection.

In one embodiment, the panel additionally comprises CXCL10 (IP-10).

Thus, according to a further aspect of the invention, there is provided the use of a panel comprising the biomarkers IFN gamma, IL-10, CD120b, CD14 and CXCL10 (IP-10) for diagnosing and/or monitoring the progression of an active mycobacterial infection. A panel according to this aspect of the invention is referred to herein as the 5BM panel.

Data is presented in Tables 4 and 5 and FIGS. 2, 3 and 5 which shows the ability of the 5BM panel to identify those patients with active tuberculosis with high sensitivity and specificity.

Thus, it will be appreciated that the 5BM panel according to the invention may successfully differentially diagnose between patients with an active mycobacterial infection, such as tuberculosis, and sick control or healthy control patients. Such diagnoses provide specificity, sensitivity and accuracy values that are within diagnostically useful parameters and that match or exceed the World Health Organization (WHO) targets for such tests.

IFN gamma is a dimerised soluble cytokine that in humans is encoded by the IFNG gene. IFN gamma is an important activator of macrophages and can induce MHC class II expression. The IFN gamma monomer consists of a core of six α-helices and an extended unfolded sequence in the C-terminal region. The biologically active dimer is formed by anti-parallel inter-locking of the two monomers. Cellular responses to IFN gamma are activated through its interaction with a heterodimeric receptor consisting of Interferon gamma receptor 1 (IFNGR1) and Interferon gamma receptor 2 (IFNGR2), whereupon binding activates the JAK-STAT pathway. IFN gamma also binds to the glycosaminoglycan heparan sulfate (HS) at the cell surface and the binding of IFN gamma to HS inhibits its biological activity.

IFN gamma is associated with the formation of granulomas which can be due to either infectious or non-infectious causes. Infectious causes of granulomas include tuberculosis, leprosy, histoplasmosis, cryptococcosis, coccidioidomycosis and blastomycosis. Examples of non-infectious granulomatous diseases include sarcoidosis and Crohn's disease. IFN gamma activate macrophages so they become more potent in their clearance of intracellular organisms and pathogens. Cycles of activation and IFN gamma release eventually lead to the surrounding of an infectious site by macrophages in a fibroblast-like manner.

IL-10 (Interleukin 10) also known as human cytokine synthesis inhibitory factor (CSIF), is an anti-inflammatory cytokine that in humans is encoded by the IL10 gene. IL-10 signals through a receptor complex consisting of two IL-10 receptor-1 and two IL-10 receptor-2 proteins. Consequently, the functional receptor consists of four IL-10 receptor molecules. IL-10 binding induces STAT3 signalling via the phosphorylation of the cytoplasmic tails of the receptor by JAK1 and Tyk2.

CD120b, also known as Tumour necrosis factor receptor 2 (TNFR2) and tumour necrosis factor receptor superfamily member 1B (TNFRSF1B), is a membrane receptor that binds tumour necrosis factor-alpha (TNFα).

CD14 is a component of the innate immune system and can exist in two forms, one anchored to the membrane by a glycosylphosphatidylinositol tail (mCD14), the other a soluble form (sCD14). Soluble CD14 may be released by shedding of mCD14 or is secreted from intracellular vesicles. CD14 acts as a co-receptor (along with the Toll-like receptor TLR 4 and MD-2) for the detection of bacterial lipopolysaccharide (LPS). CD14 can also recognise other pathogen-associated molecular patterns such as lipoteichoic acid.

It will be appreciated that CD14 and/or CD120b may be used in the invention either in its membrane-anchored form or its soluble form. Furthermore, such biomarkers may further be used when comprised in vesicles or membrane parts.

References herein to “CXCL10” refer to C-X-C motif chemokine 10 (CXCL10) also known as Interferon gamma-induced protein 10 (IP-10) or small-inducible cytokine B10. CXCL10 is an 8.7 kDa protein that in humans is encoded by the CXCL10 gene. CXCL10 is a small cytokine belonging to the CXC chemokine family.

It will be appreciated that CXCL10 may be used in the invention in either its native form or its soluble form (i.e. antagonist form). A discussion of these differing forms of CXCL10 is described in Casrouge et al. (2011) The Journal of Clinical Investigation 121(1), 308-317 where it is hypothesised that dipeptidyl peptidase IV (DPP4; also known as CD26), possibly in combination with other proteases, mediates the generation of the antagonist form(s) of CXCL10.

In one embodiment, the panel according to the first aspect of the invention additionally comprises at least one of IL-8, IL-12p70 and CXCL9. In a further embodiment, the panel according to the first aspect of the invention additionally comprises at least two of IL-8, IL-12p70 and CXCL9.

In a yet further embodiment, the panel according to the first aspect of the invention additionally comprises each of IL-8, IL-12p70 and CXCL9.

Thus, according to a further aspect of the invention, there is provided the use of a panel comprising the biomarkers IFN gamma, IL-10, CD120b, CD14, IL-8, IL-12p70 and CXCL9 for diagnosing and/or monitoring the progression of an active mycobacterial infection. A panel according to this aspect of the invention is referred to herein as the 7BM panel.

Data is presented in Tables 4 and 5 and FIGS. 2, 3 and 5 which shows the ability of the 7BM panel to identify those patients with active tuberculosis with high sensitivity and specificity.

Thus, it will be appreciated that the 7BM panel according to the invention may successfully differentially diagnose between patients with an active mycobacterial infection, such as tuberculosis, and sick control or healthy control patients. Such diagnoses provide specificity, sensitivity and accuracy values that are within diagnostically useful parameters and that match or exceed the World Health Organization (WHO) targets for such tests.

Interleukin 8 (IL8 or chemokine (C-X-C motif) ligand 8, CXCL8), also known as neutrophil chemotactic factor, is a chemokine produced by macrophages and other cell types such as epithelial cells and in humans is encoded by the CXCL8 gene. IL-8 is initially produced as a precursor peptide of 99 amino acids which then undergoes cleavage to create several active IL-8 isoforms. A 72 amino acid peptide is the major form secreted by macrophages in culture. Multiple cell surface receptors are capable of binding IL-8, however the most well-known are CXCR1 and CXCR2. In target cells, IL-8 induces a series of physiological responses required for migration and phagocytosis, such as increases in intracellular Ca²⁺ and exocytosis.

IL-12p70 is the active heterodimer of p35 and p40 subunits, encoded for in humans by the IL12A and IL12B genes respectively. IL-12 is involved in the stimulation, growth and function of T cells and can stimulate the production of IFN gamma and TN Fa. IL-12 plays an important role in the activities of natural killer cells and T lymphocytes and there is evidence of a link between IL-2 and the signal transduction of IL-12 in NK cells. IL-2 stimulates the expression of two IL-12 receptors, IL-12R-β1 and IL-12R-β2, maintaining the expression of a critical protein involved in IL-12 signalling in NK cells.

Chemokine (C-X-C motif) ligand 9 (CXCL9) is a small cytokine belonging to the CXC chemokine family that is also known as Monokine induced by gamma interferon (MIG) and is a T-cell chemoattractant, which is induced by IFN gamma. It is closely related to CXCL10 and CXCL11 and elicits its function by interacting with the chemokine receptor CXCR3.

According to a further aspect of the invention, there is provided a method of diagnosing the absence of an active mycobacterial infection in an individual, comprising:
(a) obtaining a test biological sample from an individual;
(b) quantifying the amount of the biomarkers as defined herein;
(c) inputting said amounts into a prediction model generated by an algorithm which comprises the relative amounts of each biomarker in active mycobacterial infected patients;
(d) obtaining a likelihood value for the absence of an active mycobacterial infection from said model.

It will be appreciated that, according to certain aspects of the invention, the absence of an active mycobacterial infection may be indicative of the presence of a latent mycobacterial infection or the absence of a mycobacterial infection. Alternatively or additionally, the absence of an active mycobacterial infection may be indicative of the presence of a non-mycobacterial infection.

In one embodiment, said individual is a mammal. In a further embodiment, said mammal is a human, such as a human individual or a human subject.

According to a further aspect of the invention, there is provided a method of excluding the presence, and/or monitoring the progression of, an active mycobacterial infection in an individual thereto, comprising:
(a) obtaining a test biological sample from an individual;
(b) quantifying the amount of the biomarkers as defined herein;
(c) comparing the amounts of the biomarkers in the test biological sample with the amounts present in one or more control samples, such that a difference in the level of the biomarkers in the test biological sample is indicative of the absence of an active mycobacterial infection.

It will be appreciated that, according to certain aspects of the invention, excluding the presence of an active mycobacterial infection includes identifying an absence of an active infection or the presence of a latent mycobacterial infection. Furthermore, excluding the presence of an active mycobacterial infection may include the diagnosis of a latent mycobacterial infection. Alternatively or additionally, excluding the presence of an active mycobacterial infection may comprise the identification of a non-mycobacterial infection.

It will be further appreciated that, according to aspects of the invention, monitoring the progression of an active mycobacterial infection includes the monitoring of the progression of a latent mycobacterial infection to an active mycobacterial infection. Furthermore, such monitoring aspects may also include the determination of the likelihood of progression of a latent mycobacterial infection to an active mycobacterial infection.

According to a further aspect of the invention, there is provided a method of determining the efficacy of anti-mycobacterial therapy for an active mycobacterial infection in an individual subject comprising:
(a) obtaining a biological sample from an individual;
(b) quantifying the amount of the biomarkers as defined herein;
(c) comparing the amounts of the biomarkers in the test biological sample with the amounts present in one or more control samples, such that a difference in the level of the biomarkers in the test biological sample is indicative of a response to the treatment.

In one embodiment of the invention, the biological samples obtained from an individual may be taken on two or more occasions. In a further embodiment, the individual may be a test subject or a control subject.

In one embodiment, the method as defined herein further comprises detecting a change in the amount of the biomarkers in samples taken on two or more occasions.

It will be appreciated that references herein to diagnosing, excluding the presence of, monitoring the progression of, diagnosing the absence of or treating an active mycobacterial infection in an individual include references to said individual being co-infected with HIV.

It will be further appreciated that references herein to diagnosing, excluding the presence of, monitoring the progression of, diagnosing the absence of or treating an active mycobacterial infection in an individual include individuals from all ethnicities and backgrounds.

The biomarker panels of the invention have the potential to provide a number of key advantages over the existing diagnostic tests for tuberculosis. These include rapid diagnosis from serological samples which are relatively easily obtained with no requirement for overnight incubation with disease specific antigens. This is of particular significance in cases of extrapulmonary TB and in paediatric cases. It also enhances the potential for TB diagnosis in the absence of Category 3 laboratory provisions. The data also supports enhanced diagnostic capability in immunocompromised individuals. Current immunologically based diagnostic adjuncts including the IGRAs, have a low sensitivity and specificity in diagnosing tuberculosis in HIV infected individuals, primarily because HIV-infected TB patients produce quantitatively less gamma interferon in response to TB-specific antigens than HIV-negative TB patients (Tsiouris et al. (2006) J Clin Microbiol. 44(8): 2844-2850). By contrast, the biomarkers of the invention are expected to have great utility in the diagnosis of tuberculosis (including extrapulmonary tuberculosis) in immunocompromised individuals by virtue of high levels of specificity and sensitivity. The biomarker panels also provide more sensitive and specific discriminations between key clinical discriminations of patient groups.

Thus, in some embodiments, the present invention may be utilised for the diagnosis, excluding the presence of, monitoring the progression of, diagnosing the absence of or treating an active mycobacterial infection in immunocompromised or HIV positive individuals. However, it will also be appreciated that the determination of an individual's HIV status does not need to be performed in order for the panels of the present invention to diagnose, monitor the progression of, diagnose the absence of or for the treatment of an active mycobacterial infection.

In further embodiments, the present invention may utilise and/or include clinical data or information, such as the HIV status of a patient. Thus, in one embodiment, the biomarker panels of the invention further comprise HIV status.

Furthermore, in some embodiments the present invention may be utilised for the diagnosis, excluding the presence of, monitoring the progression of, diagnosing the absence of or treatment of an active mycobacterial infection in paediatric patients or those otherwise unable to produce a sputum or bronchiolar lavage sample. Thus, it will be appreciated that the panels of the present invention can be used for the diagnosis, excluding the presence of, monitoring the progression of, diagnosing the absence of or for the treatment of an extrapulmonary active mycobacterial infection, such as extrapulmonary tuberculosis.

The biomarker panels of the invention also provide the potential for greater comprehension of the immunological response to the disease, and therefore the development of targeted immunotherapy both in respect to the development of immunotherapy for active disease, and the development of post-exposure prophylaxis.

In one embodiment, the active mycobacterial infection is active tuberculosis.

References herein to “tuberculosis” include an infectious disease caused by the presence of the pathogen Mycobacterium tuberculosis. References to tuberculosis also include references to active symptomatic tuberculosis infection and latent asymptomatic tuberculosis infection (LTBI). Most instances of tuberculosis infection are primarily restricted to the lungs (i.e. pulmonary tuberculosis), however, approximately one quarter of active tuberculosis infections move from the lungs, causing other kinds of tuberculosis, collectively referred to as extrapulmonary tuberculosis. This occurs more commonly in immunosuppressed persons (i.e. those suffering from HIV) and young children.

In one embodiment, the active mycobacterial infection is extrapulmonary tuberculosis.

In one embodiment, the diagnosis or excluding the presence of a mycobacterial infection is differential diagnosis between:
(i) active tuberculosis patients and patients with latent, non-progressing tuberculosis or sick patients or healthy patients; and/or
(ii) the patient groups of (i), irrespective of whether they have been characterised as being smear positive or smear negative; and/or
(iii) the patient groups of (i) and/or (ii), irrespective of whether they have been characterised as being HIV positive or HIV negative.

The term “biomarker” refers to a distinctive biological or biologically derived indicator of a process, event, or condition. Biomarkers can be used in methods of diagnosis, e.g. clinical screening, and prognosis assessment, prediction of active mycobacterial disease development (for example from latent infection) and in monitoring the results of therapy, identifying patients most likely to respond to a particular therapeutic treatment, drug screening and development. Biomarkers and uses thereof are valuable for identification of new drug treatments and for discovery of new targets for drug treatment. In particular, the biomarker panels of the invention have the potential to effectively monitor the immunological response to anti-mycobacterial therapy. For example, it could be easily established which patients have the potential for a shortened course of treatment (currently tuberculosis treatment for sensitive strains ranges from 6 to 12 months). They may also form a useful predictive signature which predicts those who will progress to active disease, thereby allowing targeted treatment of a subgroup of latently infected individuals who have a high chance of progressing to active disease.

In view of the fact that the invention relates to the diagnosis, excluding the presence of, monitoring of the progression of or diagnosis of the absence of an infectious disease, drug resistant mutations of mycobacteria are of particular concern. For example, certain strains of tuberculosis exist which are resistant to particular forms of anti-tuberculosis treatment, such as rifampicin resistant tuberculosis. The above mentioned “monitoring” aspects of the invention are therefore of critical importance because non-response to treatment can be an early sign that the tuberculosis may be rifampicin or multi-drug resistant tuberculosis. In this situation, alternative treatment regimens may be employed at a much earlier phase which will allow a greater possibility for the treated individual to survive and decrease transmission.

In one embodiment, one or more of the biomarkers may be replaced by a molecule, or a measurable fragment of the molecule, found upstream or downstream of the biomarker in a biological pathway.

In one embodiment, a prediction model is generated by an algorithm which comprises the relative amounts of each biomarker. Amounts of biomarkers quantified according to the invention may then be inputted into said prediction model and a likelihood value for the presence or absence of an active mycobacterial infection obtained.

In one embodiment, the detecting and/or quantifying of the biomarkers as defined herein is performed by measuring the concentration of the analyte biomarkers in each sample. In a further embodiment, the detecting and/or quantifying of the biomarkers as defined herein is performed using an immunological method.

It will be appreciated that, in one embodiment, the detecting and/or quantifying of the biomarkers as defined herein is performed using a biosensor or a microanalytical, microengineered, microseparation or immunochromatography system.

As used herein, the term “biosensor” means anything capable of detecting the presence of the biomarker. Examples of biosensors are described herein.

Biosensors according to the invention may comprise a ligand or ligands, as described herein, capable of specific binding to the biomarker. Such biosensors are useful in detecting and/or quantifying a biomarker or biomarker panel according to the invention.

Diagnostic kits for the diagnosis, excluding the presence of and monitoring of tuberculosis are described herein. In one embodiment, the kits additionally contain a biosensor capable of detecting and/or quantifying a biomarker.

Detection and/or quantification of biomarkers may be performed by detection of the biomarker or of a fragment thereof, e.g. a fragment with C-terminal truncation, or with N-terminal truncation. Fragments are suitably greater than 4 amino acids in length, for example 5, 6, 7, 8, 9, 10, 11, 12, 13, 14, 15, 16, 17, 18, 19, or 20 amino acids in length.

The biomarker may be directly detected, e.g. by SELDI or MALDI-TOF. Alternatively, the biomarker may be detected directly or indirectly via interaction with a ligand or ligands such as an antibody or a biomarker-binding fragment thereof, or other peptide, or ligand, e.g. aptamer, or oligonucleotide, capable of specifically binding the biomarker. The ligand may possess a detectable label, such as a luminescent, fluorescent or radioactive label, and/or an affinity tag.

For example, detecting and/or quantifying can be performed by one or more method(s) selected from the group consisting of: SELDI (-TOF), MALDI (-TOF), a 1-D gel-based analysis, a 2-D gel-based analysis, Mass spectrometry (MS), reverse phase (RP) LC, size permeation (gel filtration), ion exchange, affinity, HPLC, UPLC and other LC or LC MS-based techniques. Appropriate LC MS techniques include ICAT® (Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

Methods of diagnosing, and/or monitoring according to the invention may comprise analysing a plasma, serum or whole blood sample by a sandwich immunoassay to detect the presence or level of the biomarker. These methods are also suitable for clinical screening, prognosis, monitoring the results of therapy, identifying patients most likely to respond to a particular therapeutic treatment, for drug screening and development, and identification of new targets for drug treatment.

Detecting and/or quantifying the biomarkers may be performed using an immunological method, involving an antibody, or a fragment thereof capable of specific binding to the biomarker. Suitable immunological methods include sandwich immunoassays, such as sandwich ELISA, in which the detection of the biomarkers is performed using two antibodies which recognize different epitopes on a biomarker; radioimmunoassays (RIA), direct, indirect or competitive enzyme linked immunosorbent assays (ELISA), enzyme immunoassays (EIA), Fluorescence immunoassays (FIA), western blotting, immunoprecipitation and any particle-based immunoassay (e.g. using gold, silver, or latex particles, magnetic particles, or Q-dots). Immunological methods may be performed, for example, in microtitre plate or strip format.

Immunological methods in accordance with the invention may be based, for example, on the following methods.

Enzyme (EIA) immunoassays were developed as an alternative to radioimmunoassays (RIA). These methods use an enzyme to label either the antibody or target antigen. The sensitivity of EIA approaches that for RIA, without the danger posed by radioactive isotopes. One of the most widely used EIA methods for detection is the enzyme-linked immunosorbent assay (ELISA). ELISA methods may use two antibodies one of which is specific for the target antigen and the other of which is coupled to an enzyme, addition of the substrate for the enzyme results in production of a chemiluminescent or fluorescent signal.

Fluorescent immunoassay (FIA) refers to immunoassays which utilize a fluorescent label or an enzyme label which acts on the substrate to form a fluorescent product. Fluorescent measurements are inherently more sensitive than colorimetric (spectrophotometric) measurements. Therefore, FIA methods have greater analytical sensitivity than EIA methods, which employ absorbance (optical density) measurement.

Chemiluminescent immunoassays utilize a chemiluminescent label, which produces light when excited by chemical energy; the emissions are measured using a light detector.

Immunological methods according to the invention can thus be performed using well-known methods. Any direct (e.g., using a sensor chip) or indirect procedure may be used in the detection of biomarkers of the invention.

The Biotin-Avidin or Biotin-Streptavidin systems are generic labelling systems that can be adapted for use in immunological methods of the invention. One binding partner (hapten, antigen, ligand, aptamer, antibody, enzyme etc) is labelled with biotin and the other partner (surface, e.g. well, bead, sensor etc) is labelled with avidin or streptavidin. This is conventional technology for immunoassays, gene probe assays and (bio)sensors, but is an indirect immobilisation route rather than a direct one. For example, a biotinylated ligand (e.g. antibody or aptamer) specific for a biomarker of the invention may be immobilised on an avidin or streptavidin surface, the immobilised ligand may then be exposed to a sample containing or suspected of containing the biomarker in order to detect and/or quantify a biomarker of the invention. Detection and/or quantification of the immobilised antigen may then be performed by an immunological method as described herein.

In alternative embodiments, the ligand (e.g. antibody or aptamer) may be bound to a surface (e.g. through biotin-streptavidin binding as described above or directly bound to the surface) and detection and/or quantification of the immobilised antigen may be performed directly without an additional detection step (e.g. by surface plasmon resonance).

The term “antibody” as used herein includes, but is not limited to: polyclonal, monoclonal, bispecific, humanised or chimeric antibodies, single chain antibodies, fragments (such as FAb, F(Ab′)₂, Fv, disulphide linked Fv, scFv, diabody), fragments produced by a Fab expression library, anti-idiotypic (anti-Id) antibodies and epitope-binding fragments of any of the above. The term “antibody” as used herein also refers to immunoglobulin molecules and immunologically-active portions of immunoglobulin molecules, i.e., molecules that contain an antigen binding site that specifically binds an antigen. The immunoglobulin molecules of the invention can be of any class (e. g., IgG, IgE, IgM, IgD and IgA) or subclass of immunoglobulin molecule.

The identification of key biomarkers specific to a disease is central to integration of diagnostic procedures and therapeutic regimes. Using predictive biomarkers appropriate diagnostic tools such as biosensors can be developed; accordingly, in methods and uses of the invention, detecting and quantifying can be performed using a biosensor, microanalytical system, microengineered system, microseparation system, immunochromatography system or other suitable analytical devices. The biosensor may incorporate an immunological method for detection of the biomarker(s), electrical, thermal, magnetic, optical (e.g. hologram) or acoustic technologies. Using such biosensors, it is possible to detect the target biomarker(s) at the anticipated concentrations found in biological samples.

Thus, according to a further aspect of the invention there is provided an apparatus for diagnosing and/or monitoring tuberculosis which comprises a biosensor, microanalytical, microengineered, microseparation and/or immunochromatography system configured to detect and/or quantify any of the biomarkers defined herein.

Suitably, biosensors for detection of one or more biomarkers of the invention combine biomolecular recognition with appropriate means to convert detection of the presence, or quantitation, of the biomarker in the sample into a signal. Biosensors can be adapted for “alternate site” diagnostic testing, e.g. in the ward, outpatients' department, surgery, home, field and workplace.

Biosensors to detect one or more biomarkers of the invention include acoustic, plasmon resonance, holographic and microengineered sensors. Imprinted recognition elements, thin film transistor technology, magnetic acoustic resonator devices and other novel acousto-electrical systems may be employed in biosensors for detection of the one or more biomarkers of the invention.

Levels of the biomarkers of the invention may be measured in accordance with any of the techniques described hereinbefore. In one embodiment, the biomarkers are detected and/or measured using an indirect sandwich immunoassay. The term “indirect” refers to the fact that the detection antibody is not conjugated directly to a label meaning that a third-step is required to attach a label to the detection antibody. Streptavidin-biotin may be used for attachment of label to detection antibody. Use of streptavidin-biotin allows the use of antibodies which have been biotinylated by the manufacturer in combination with commercially available streptavidin-tag conjugates. It is possible to reduce steps in assay and time-to-w result by directly labelling the detection antibody with a label (e.g. a chemiluminescent of fluorescent label).

Monitoring methods of the invention can be used to monitor onset, progression, stabilisation, amelioration, remission and/or response to therapeutic intervention or of the active mycobacterial infection. It will also be appreciated that monitoring may also include monitoring the extent of mycobacterial infection to detect the severity of the disease. The markers of the invention may provide differentiation between latent mycobacterial infection and active mycobacterial infection. For example, the invention finds great utility in assisting diagnostic capability both in terms of latent disease, active disease and establishing those with latent disease that may progress to active disease.

In methods of diagnosis, excluding the presence of, monitoring of the progression of or diagnosis of the absence of an infectious disease according to the invention, detecting and/or quantifying the biomarker in a biological sample from a test subject may be performed on two or more occasions. Comparisons may be made between the level of biomarker in samples taken on two or more occasions. Assessment of any change in the level of the biomarker in samples taken on two or more occasions may be performed. Modulation of the biomarker level is useful as an indicator of the state of mycobacterial infection. A decrease in the level of the biomarker, over time may be indicative of onset or progression, i.e. worsening of the disorder, whereas an increase in the level of the biomarker indicates amelioration or remission of the disorder, or vice versa.

A method of diagnosis, excluding the presence of, monitoring of the progression of or diagnosis of the absence of an infectious disease according to the invention may comprise quantifying the biomarker in a test biological sample from a test subject and comparing the level of the biomarker present in said test sample with one or more controls.

The control used in any one of the methods of the invention defined herein may comprise one or more control samples selected from: the level of biomarker found in a healthy control sample from a healthy individual, a healthy biomarker level; or a healthy biomarker range; patients with other respiratory infections; patients with non-mycobacterial infections; patients with non-TB mycobacterial infections; and patients known to have active or latent TB.

For biomarkers which are increased in patients with active mycobacterial infection, a higher level of the biomarker in the test sample relative to the level in the healthy control is indicative of a diagnosis of an active mycobacterial infection; an equivalent or lower level of the biomarker in the test sample relative to the healthy control is indicative of absence of an active mycobacterial infection. For biomarkers which are decreased in patients with active mycobacterial infection, a lower level of the biomarker in the test sample relative to the level in the healthy control is indicative of the diagnosis of an active mycobacterial infection; an equivalent or lower level of the biomarker in the test sample relative to the healthy control is indicative of absence of an active mycobacterial infection. It will also be appreciated that wherein the control sample comprises a sample obtained from a patient with active or latent mycobacterial infection, a positive diagnosis of active or latent mycobacterial infection will typically require a substantially similar level of the biomarker with the control sample.

The term “diagnosis” as used herein encompasses identification, confirmation, characterisation, identification of the absence and/or exclusion of the presence of active mycobacterial infection. Methods of monitoring and of diagnosis according to the invention are useful to confirm the existence of an active mycobacterial infection; to monitor development of the disorder by assessing onset and progression, or to assess amelioration or regression of the disorder. Methods of monitoring and of diagnosis are also useful in methods for assessment of clinical screening, prognosis, choice of therapy, evaluation of therapeutic benefit, i.e. for drug screening and drug development.

Efficient diagnosis and monitoring methods provide very powerful “patient solutions” with the potential for improved prognosis, by establishing the correct diagnosis, allowing rapid identification of the most appropriate treatment (thus lessening unnecessary exposure to harmful drug side effects). In a particular embodiment, the diagnosis of the absence of an active mycobacterial infection prevents the unnecessary exposure to harmful drug side effects.

In a further embodiment, the methods of the invention as defined herein are performed using a biological sample selected from whole blood, serum, plasma, tissue fluid, cerebrospinal fluid (CSF), synovial fluid, follicular fluid, seminal fluid, amniotic fluid, milk, urine, pleural fluid, ascites, bronchoalveolar lavage, saliva, sputum, tears, perspiration, lymphatic fluid, aspirate, bone marrow aspirate and mucus, or an extract or purification therefrom, or dilution thereof. In a particular embodiment, the methods of the invention as defined herein are performed using a biological sample selected from whole blood, serum or plasma. In a particular embodiment, the methods of the invention as defined herein are performed using a biological sample of serum.

It will be appreciated that embodiments of the methods as defined herein include the use of serum such as non-activated serum and/or serum that has been activated using one or more disease specific antigens, such as Mycobacterium tuberculosis specific antigens or in the case of diagnosis of other mycobacterial diseases with the relevant disease specific antigens.

Thus, according to a further aspect of the invention, there is provided a method of excluding the presence, monitoring the progression or diagnosing the absence of an active mycobacterial infection, active tuberculosis or extrapulmonary tuberculosis comprising:
(a) obtaining a serum sample from an individual;
(b) dividing said serum sample into a serum sample for activation using disease specific antigens, such as Mycobacterium tuberculosis specific antigens, and a non-activated serum sample;
(c) activating said serum sample for activation using disease specific antigens, such as Mycobacterium tuberculosis specific antigens;
(d) quantifying the amount of the biomarkers as defined herein in each of the activated and non-activated serum samples;
(e) comparing the amounts of the biomarkers in the activated serum sample with the amounts present in the non-activated serum sample, such that a difference in the level of the biomarkers in the activated serum sample is indicative of:
   (i) a diagnosis of tuberculosis; and/or
   (ii) the presence of tuberculosis infection rather than any other mycobacterial infection.

Mycobacterium tuberculosis specific antigens which can be used according to a method of the invention as defined herein are known in the art. Such M. tuberculosis specific antigens may include, without limitation, ESAT-6 and other ESAT proteins, CFP-10, and ESPC or fragments or derivatives thereof. In a further embodiment, activation of a serum sample may be performed using inactivated M. tuberculosis or portions or fragments thereof or tuberculin purified protein derivative (PPD).

According to a further aspect of the invention, there is provided a method of treating an active mycobacterial infection in an individual in need thereof, wherein said method comprises the following steps:
(a) excluding the presence, and/or monitoring the progression of, an active mycobacterial infection in an individual according to the methods defined herein; followed by
(b) administering an anti-mycobacterial medicament to said individual in the event of a positive diagnosis for active mycobacterial infection.

It will be appreciated that a method according to this aspect of the invention may also utilise the diagnosis of the absence of exclusion of the presence of an active mycobacterial infection to determine the appropriate treatment of said individual. In some embodiments, the diagnosis of the absence or exclusion of the presence of an active mycobacterial infection may determine that administration of an anti-mycobacterial medicament is not appropriate.

In a further embodiment, the active mycobacterial infection is active tuberculosis or extrapulmonary tuberculosis and the anti-mycobacterial medicament is an anti-tuberculosis medicament.

Such anti-tuberculosis medicaments are known in the art and may include, without limitation: one or more first line medicaments selected from: ethambutol, isoniazid, pyrazinamide, rifampicin; and/or one or more second line medicaments selected from: aminoglycosides (e.g., amikacin, kanamycin), polypeptides (e.g., capreomycin, viomycin, enviomycin), fluoroquinolones (e.g., ciprofloxacin, levofloxacin, moxifloxacin), thioamides (e.g. ethionamide, prothionamide), cycloserine (closerin) or terizidone; and/or one or more third line medicaments selected from rifabutin, macrolides (e.g., clarithromycin), linezolid, thioacetazone, thioridazine, arginine, vitamin D and R207910.

According to a further aspect of the invention, there is provided a kit capable of detecting and/or quantifying the amount of the biomarkers as defined herein, for use in diagnosing, excluding the presence of, monitoring the progression of and/or diagnosing the absence of an active mycobacterial infection. Suitably a kit according to the invention may contain one or more components selected from the group: a ligand specific for the biomarker or a structural/shape mimic of the biomarker, one or more controls, one or more reagents and one or more consumables; optionally together with instructions for use of the kit in accordance with any of the methods defined herein. In one embodiment, the kit diagnoses the absence of an active mycobacterial infection, such as active tuberculosis.

In one embodiment of the kit defined herein, the active mycobacterial infection is active tuberculosis. In a further embodiment of the kit defined herein, the active mycobacterial infection or active tuberculosis is extrapulmonary tuberculosis.

The identification of biomarker panels for active mycobacterial infections permits integration of diagnostic procedures and therapeutic regimes. Currently there are significant delays in determining effective treatment and hitherto it has not been possible to perform rapid assessment of drug response. Traditionally, many tuberculosis therapies have required treatment trials lasting weeks to months for a given therapeutic approach. Detection of a biomarker panel of the invention can be used to screen subjects prior to their participation in clinical trials. The biomarkers provide the means to indicate therapeutic response, failure to respond, unfavourable side-effect profile, degree of medication compliance and achievement of adequate serum or plasma drug levels. The biomarkers may be used to provide warning of adverse drug response. Biomarkers are useful in development of personalized therapies, as assessment of response can be used to fine-tune dosage, minimise the number of prescribed medications, reduce the delay in attaining effective therapy and avoid adverse drug reactions. Thus, by monitoring a biomarker of the invention, patient care can be tailored precisely to match the needs determined by the disorder and the pharmacogenomic profile of the patient, the biomarker can thus be used to titrate the optimal dose, predict a positive therapeutic response and identify those patients at high risk of severe side effects.

Biomarker panel-based tests provide a first line assessment of ‘new’ patients, and provide objective measures for accurate and rapid diagnosis, in a time frame and with precision, not achievable using the current subjective measures.

Biomarker monitoring methods, biosensors and kits are also vital as patient monitoring tools, to enable the physician to determine whether relapse is due to worsening of the disorder, poor patient compliance or drug resistance. In particular, the invention may also be used to monitor patient compliance with taking a particular drug (agent), and/or undergoing a particular treatment regime.

If pharmacological treatment is assessed to be inadequate, then therapy can be reinstated or increased; a change in therapy can be given if appropriate. As the biomarkers are sensitive to the state of the disorder, they provide an indication of the impact of drug therapy.

The following studies illustrate the invention.

### Materials and Methods

Samples were obtained from the Foundation for Innovative New Diagnostics (FIND, Geneva, Switzerland), IGRA in Diagnostic Evaluation of Active TB (IDEA) study and from patients in South Africa as detailed below.

### Find

Since 2003, the Foundation for Innovative New Diagnostics (FIND, Geneva, Switzerland) has curated a high quality, geographically diverse specimen bank to support the development of TB diagnostic tests.

Through a Material Transfer Agreement of October 2014, a large number of serum samples sourced from Vietnam, Peru and South Africa were graciously made available by FIND for the work presented here.

The FIND samples were from enrolled adults who presented with signs and symptoms of TB (cough for at least 2 weeks, fevers, weight loss, and night sweats). Basic demographics, such as age, weight, and gender, and clinical metadata, such as HIV status and, in some cases, CD4 cell counts and viral load, were collected. Subjects with culture positivity were considered confirmed TB cases. Criteria for ruling out TB were defined by FIND and included culture and smear negativity and resolution of symptoms in the absence of specific TB therapy at follow-up at 2 to 3 months.

### Idea (London)

A prospective multi-centre cohort study was conducted in the UK (in routine clinical practice) of >1,012 adults 16 years old, of which the present inventors had access to 816 samples) presenting with suspected active tuberculosis at either NHS out-patient or in-patient services within participating hospitals in London, Slough, Leicester, Birmingham and Oxford.

Patients were recruited at the point of diagnostic work-up in secondary care, before a confirmed diagnosis was determined. Thus, potential participants for the study were identified at this point. The study population is representative of the national tuberculosis burden in terms of ethnic mix and range of co-morbidities and includes sufficient numbers of HIV-infected patients to reliably determine the role of IGRAs in this key population.

Blood taking (venepuncture) procedures were carried out during the study period and undertaken in accordance with local venepuncture guidelines. Visits and blood samples were taken at the same time as a participant's routine clinic appointment.

The frequency and timing of blood taking procedures was at three set time intervals. The first sample, at time zero (TO) was no later than 14 days after the start of treatment for TB or within 7 days of consent, whichever occurred sooner, depending on the patient's diagnosis. Follow-ups, in addition to the set time intervals, were carried out, if clinically indicated. A 35 ml blood sample was taken at each time point and used testing and for storage (PBMCs and serum) in the bio-repository.

A 35 ml quantity specimen of blood was collected from all consenting participants for testing; residual sample was stored to repeat the small number of indeterminate results expected and for future research. PBMCs were stored in a liquid nitrogen tank and serum and plasma stored in a −80° C. freezer at the Tuberculosis Research Unit at Imperial College London. On the same day as sample collection, blood samples were transported in appropriate UN-type approved packaging to the TB Research Unit at Imperial College London (St. Mary's Hospital), for testing.

In patients with sputum smear negative pulmonary TB, diagnostic bronchoscopes were performed and collection of BAL (BronchoAlveolar Lavage) obtained as part of routine clinical care. In cases where an amount of BAL sample was left over after the procedure, an aliquot from the sample was cryopreserved and stored in the research bio-repository.

Samples of cultures positive for M. tuberculosis grown from diagnostic specimens were collected from the participating site's clinical diagnostic laboratory. These samples were stored in order to subsequently make correlations between different clinical phenotypes of TB disease and characteristics of the infecting mycobacterial strains.

Participants were classified into the final diagnosis reference standard categories defined by Dosanjh et al. (Ann Intern Med. 2008 Mar. 4; 148(5):325-36).

### Stratification of Patient Samples—IDEA Cohort

Independent training (570 patient samples) and test (246 patient samples) datasets were blindly chosen from the IDEA cohort. The same diagnostic grouping was used as previously by other authors (Dosanjh et al. (2008) Annals of Internal Medicine 148:325) 1=active TB smear positive, 2=active TB smear negative, 4A=latent TST positive exposure positive, 4B=latent TST positive exposure negative, 4C=non-TB TST negative exposure positive and 4D=non-TB TST negative exposure negative. The groups were split into HIV positive and HIV negative samples. Data is presented in Table 1, below.

TABLE 1
Stratification of patient samples-IDEA cohort:

| 570 training samples | | | HIV −ve | | HIV +ve | |
|---|---|---|---|---|---|---|
| 246 test samples | | | Train | Test | Train | Test |
| 1 | active TB | smear +ve | 37.5% | 37.2% | 9.2% | 10.6% |
| | | | (173) | (74) | (10) | (5) |
| 2 | | smear −ve | 13.2% | 13.1% | 9.2% | 8.5% |
| | | | (61) | (26) | (10) | (4) |
| 4A | latent | exposure +ve | 7.3% | 7.5% | 15.6% | 17.0% |
| | TST +ve | | (34) | (15) | (17) | (8) |
| 4B | | exposure −ve | 5.2% | 5.5% | 0.9% | 0% |
| | | | (24) | (11) | (1) | (0) |
| 4C | nonTB | exposure +ve | 27.8% | 27.6% | 32.1% | 44.6% |
| | TST −ve | | (128) | (55) | (35) | (21) |
| 4D | | exposure −ve | 8.9% | 9.0% | 33.0% | 19.1% |
| | | | (41) | (18) | (36) | (9) |

The percentage contribution to the respective set is shown and the absolute number of samples is given in brackets.

### Ethnical Diversity of IDEA Cohort

The number of patient samples from different ethnicities is shown in Table 2 for both the training and test datasets from the IDEA cohort. The samples were chosen at random without any consideration of any TB relevant diagnosis.

TABLE 2
Ethnical Diversity of IDEA cohort:

| | Train | Test |
|---|---|---|
| AFGHANISTAN | 4 | 1 |
| ALGERIA | 1 | 1 |
| ANTIGUA AND BARBUDA | 1 | 0 |
| ARGENTINA | 1 | 0 |
| BANGLADESH | 12 | 4 |
| BELARUS | 1 | 0 |
| BELGIUM | 0 | 1 |
| BOLIVIA | 1 | 0 |
| BRAZIL | 3 | 2 |
| BURUNDI | 1 | 1 |
| CAMEROON | 0 | 2 |
| COLOMBIA | 1 | 0 |
| CONGO | 2 | 1 |
| COTE D′IVOIRE | 1 | 0 |
| CYPRUS | 2 | 1 |
| DENMARK | 1 | 0 |
| DJIBOUTI | 0 | 1 |
| DOMINICA | 1 | 0 |
| ECUADOR | 1 | 0 |
| EGYPT | 1 | 0 |
| ENGLAND | 10 | 3 |
| ERITREA | 6 | 3 |
| ETHIOPIA | 6 | 4 |
| FRANCE | 1 | 0 |
| GAMBIA, THE | 3 | 0 |
| GERMANY | 1 | 1 |
| GHANA | 4 | 3 |
| GRENADA | 1 | 0 |
| GUINEA-BISSAU | 0 | 1 |
| HONG KONG | 0 | 1 |
| INDIA | 151 | 73 |
| INDONESIA | 1 | 0 |
| IRAN | 3 | 2 |
| IRAQ | 3 | 2 |
| IRELAND | 10 | 3 |
| ITALY | 1 | 0 |
| JAMAICA | 7 | 1 |
| JAPAN | 0 | 1 |
| KAZAKHSTAN | 1 | 0 |
| KENYA | 20 | 9 |
| KUWAIT | 2 | 0 |
| LEBANON | 0 | 1 |
| LIBYA | 1 | 0 |
| LITHUANIA | 0 | 1 |
| MALAYSIA | 1 | 0 |
| MALAWI | 4 | 0 |
| MAURITIUS | 1 | 0 |
| MOROCCO | 3 | 1 |
| MOZAMBIQUE | 1 | 0 |
| NEPAL | 10 | 6 |
| NIGER | 0 | 1 |
| NIGERIA | 8 | 7 |
| PAKISTAN | 41 | 19 |
| PHILIPPINES | 12 | 4 |
| POLAND | 3 | 6 |
| PORTUGAL | 5 | 1 |
| ROMANIA | 5 | 1 |
| SAUDI ARABIA | 1 | 0 |
| SCOTLAND | 1 | 1 |
| SOMALIA | 27 | 7 |
| SOUTH AFRICA | 6 | 3 |
| SPAIN | 1 | 0 |
| SRI LANKA | 18 | 8 |
| ST LUCIA | 1 | 0 |
| SUDAN | 4 | 1 |
| SWAZILAND | 1 | 0 |
| SWEDEN | 0 | 1 |
| SWITZERLAND | 1 | 0 |
| SYRIA | 1 | 0 |
| TANZANIA, UNITED REP. OF | 2 | 0 |
| THAILAND | 3 | 0 |
| UGANDA | 7 | 0 |
| UNITED KINGDOM | 113 | 46 |
| UNITED STATES OF AMERICA | 3 | 0 |
| WALES | 1 | 0 |
| YEMEN | 0 | 1 |
| ZAMBIA | 1 | 0 |
| ZIMBABWE | 17 | 8 |

### South Africa Samples

The study was conducted in Cape Town within high burden, developing communities with high HIV and TB prevalence at their associated primary care TB clinics. The following sites were used for patient recruitment after obtaining relevant approvals from the City of Cape Town:
Langa clinic
Gugulethu clinic
Weltevreden clinic
Vuyani clinic
Phumlani clinic

Patients, who presented to the above-mentioned clinics with respiratory symptoms, were recruited to participate in the study by a designated study nurse. Healthy volunteers were also recruited from the above mentioned communities.

At baseline, trained study personnel obtained written informed consent from the prospective participants. The participants were then screened using the inclusion and exclusion criteria to assess eligibility for enrolment into the study.

The participants' HIV status was recorded. If it was unknown, participants were counselled and tested for HIV using a rapid diagnostic kit as per the national guidelines. Approximately 25 μl of blood was obtained by finger prick. A positive rapid test result was confirmed by using a second finger-prick test. Any discordant results were confirmed by a HIV ELISA test (approximately 5 ml of blood is needed in this case). Post-test counselling was done and any newly diagnosed HIV infected participant referred to their nearest ARV facility for further counselling and management.

Healthy volunteers and symptomatic patients with a negative GeneXpert/culture result were sent for a chest X-ray.

The following samples were then collected from participants:
20 ml of blood—serum was obtained by centrifugation.
2 Sputum samples for culture, DST and storage.
1 urine sample.

The blood samples were used to test for the presence of a number of biomarkers, which represent an individual's host immune response when exposed to Mycobacteria tuberculosis.

Non-TB participants were followed up at 8 weeks post recruitment to review clinical symptoms and treatment.

### Stratification of Prospectively Collected Patient Samples—South Africa Cohort

179 prospectively collected patient samples were classified into 4 diagnostic groups. SA1 definite active TB, SA2 highly probably active TB, SA3 active TB excluded, SA4 healthy. Each diagnostic group was split into HIV +ve and HIV −ve samples as shown in Table 3. The percentage contribution of each group to the total cohort is shown. The absolute number of samples in each group are given in brackets.

TABLE 3
Stratification of prospectively collected patient samples -
South Africa cohort:

| 179 test samples | | | HIV −ve | | HIV +ve | |
|---|---|---|---|---|---|---|
| SA1 | active TB | definite | 25% | (44) | 24% | (43) |
| SA2 | | highly probable | n/a | (0) | 1% | (3) |
| SA3 | non-TB | active excluded | 29% | (52) | 10% | (18) |
| SA4 | | healthy | 11% | (19) | n/a | (0) |

Indirect sandwich immunoassays were performed as follows:

### Day 1

### Plate Coating

Purified antibodies with binding specificity to a biomarker of interest were diluted in a non-protein containing buffer and a set volume added to each of the wells of a 96-well microtitre plate. The plate was sealed and incubated at +4° C. overnight; antibodies bind to the plate via the mechanism of passive absorption.

### Day 2

### Blocking

The plate was thoroughly washed using a solution of Phosphate-buffered saline (PBS) plus 0.05% Tween-20 and blotted on a paper towel until dry.

An immunoassay blocking buffer was added to each well of the plate at a set volume which is in excess to other reagents used in the assay; this is to block any unused binding sites.

The plate was sealed and incubated for a minimum of 1 hour on a plate shaker at room temperature.

### Addition of Calibrator/QC/Test Samples

The plate was thoroughly washed using a solution of Phosphate-buffered saline (PBS) plus 0.05% Tween-20 and blotted on a paper towel until dry.

A set volume of assay diluent was added to each well of the plate.

A set volume of Calibrator/QC/Test Sample was added to each well of the plate. The use of technical replicates (running Calibrator/QC/Test Samples using duplicate or triplicate analysis) is used to understand within-batch imprecision of an assay and give confidence in the reliability of results produced.

### Calibrators

Calibrators are artificial samples produced by diluting a known amount of (recombinant) biomarker of interest in assay diluent. Serial dilutions of this material are used to produce a range of calibrator concentrations which are to be run with each batch. A blank (neat assay diluent) should be run with each calibration. The range of concentrations used for calibration should be chosen based on the concentrations which are expected to be detected in the “unknown” Test Samples. The signals measured can be plotted against the known concentrations at the end of the assay to produce a standard curve; the signals measured in the Test Samples can be read against the standard curve to provide quantitation.

### Quality Control (QC)

QC material are samples which are used to assess the accuracy and imprecision of an assay.

Multiple levels (typically 3) of QC material are run at the beginning and end of each batch. The 3 levels of QC should contain different concentrations which span the range relevant to the assay (typically low, medium and high concentrations).

QC material can be derived from pooled and aliquoted samples (such as pooled human serum or plasma) or produced by diluting recombinant biomarker in assay buffer. Multiple identical vials of each level of QC are stored at −80° C.

Running a fresh set of QC samples with every batch allows the determination of batch-to-batch imprecision.

Running multiple levels of and multiple QC samples at the beginning and end of each batch allows determination of within-batch imprecision.

Once sample loading was complete the plates were sealed and incubated for 2 hours at room temperature on a plate shaker.

### Addition of Detection Antibody

The plate was thoroughly washed using a solution of Phosphate-buffered saline (PBS) plus 0.05% Tween-20 and blotted on a paper towel until dry.

A set volume of biotinylated detection antibody was added to each well of the plate.

The plate was sealed and incubated for 1 hour on a plate shaker at room temperature.

The detection antibody should have specificity for a different epitope present on the target biomarker to the coating/capture antibody.

### Addition of Streptavidin-Tag Conjugate

The tag/label to be used is technology dependant; traditional tags for 2-site immunoassay include but are not limited to:
Enzymes (Horseradish Peroxidase)
Fluorophores (fluorescent tags)
Lanthanide metals (used in time-resolved dissociation enhanced fluorometric immunoassays)
Chemiluminescent tags
Radioactive isotopes

The plate was thoroughly washed using a solution of Phosphate-buffered saline (PBS) plus 0.05% Tween-20 and blotted on a paper towel until dry.

A set volume of streptavidin-tag conjugate was added to each well of the plate.

The plate is sealed and incubated for 30 mins on a plate shaker at room temperature.

### Read

After the addition of any reagents required to generate a signal from the tag used in the assay the plate can be read. In a two-site “sandwich” immunoassay the amount of signal generated is proportional to the concentration of biomarker in the sample.

A standard curve (generated by plotting signals measured in the serially diluted calibrator material) can be used to estimate concentrations in QC/Test Sample material.

### Ella Analysis Protocols

### 1. Simple Plex Lyophilized Quality Control Preparation

### 1.1 IFNgamma/IL-10/IL-8/CXCL-9 Multiplex

Product codes: 894955/894956/894950/894951
Lot numbers: 1509071/1498073/1393853/1394497
Re-suspension volumes (2^{nd} vials were saved for a later date) (SD13 lot #P150976)

### IFNgamma=1150 μl

### IL-10=415 μl

### IL-8=1200 μl

### CXCL-9=1000 μl

Mixed on a roller mixer for 15 minutes.

Preparation of HIGH control: In a 20 ml Universal: 360 μl of each of the control concentrates was added to 10.56 ml diluent SD13. Mixed gently.

Preparation of LOW control: In a 20 ml Universal: 240 μl of the HIGH Control was added to 11.76 ml diluent SD13.

80×140 μl aliquots prepared for the low & high QC. Freezed at −80° C.

### 1.2 IP-10/IL-12p70/IL-6/TNFa Multiplex

Product codes: 894949/894957/894968/894977
Lot numbers: 1400303/1392317/1422813/1493487
Re-suspension volumes (2^{nd} vials saved for a later date) (SD13 lot #P150976)

### IP-10=800 μl

### IL-12p70=500 μl

### IL-6=570 μl

### TNFa=600 μl

Mixed on a roller mixer for 15 minutes.

Preparation of HIGH control: In a 20 ml Universal: 360 μl of each of the control concentrates was added to 10.56 ml diluent SD13. Mixed gently.

Preparation of LOW control: In a 20 ml Universal: 240 μl of the HIGH Control was added to 11.76 ml diluent SD13.

Prepared 80×140 μl aliquots for the low & high QC. Freezed at −80° C.

### 1.3 TNF RII (CD120b) Singleplex

Product code: 898106
Lot number: 1397402
Re-suspension volume=700 μl sample diluent SD13 (product code 896098)
Add 700 μl SD13 to one vial only (2^{nd} vial saved for a later date)

### (SD13 lot #P172465)

Mixed on a roller mixer for 15 minutes.

Preparation of HIGH control: In a 20 ml Universal: 360 μl of the control concentrate was added to 11.64 ml diluent SD13. Mixed gently.

Preparation of LOW control: In a 20 ml Universal: 240 μl of the HIGH Control was added to 11.76 ml diluent SD13.

Prepared 80×140 μl aliquots for the low & high QC. Freezed at −80° C.

### 1.4 CD14 Singleplex

Product code: 898078
Lot number 1398395
Re-suspension volume=500 μl sample diluent SD13 (product code 896098)
Add 500 μl SD13 to one vial only (2^{nd} vial saved for a later date)

### (SD13 lot #P172465)

Mixed on a roller mixer for 15 minutes.

Preparation of HIGH control: In a 20 ml Universal: 360 μl of the control concentrate was added to 11.64 ml diluent SD13. Mixed gently.

Preparation of LOW control: In a 20 ml Universal: 240 μl of the HIGH Control was added to 11.76 ml diluent SD13.

Prepared 80×140 μl aliquots for the low & high QC. Freezed at −80° C.

### 2. ELLA Sample Analysis

### 2.1 ELLA Cartridges, Reagents & QCs

### 2.1.1 Cartridges

1. 72-well singleplex CD14
2. 72-well singleplex CD120b (TNFR2/TNFRSF1B)
3. 32-well 4-plex IFNgamma+IL-10+IL-8+CXCL-9
4. 32-well 4-plex IP10+IL-12p70+IL-6+TNFalpha and separately:
5. 72-well singleplex IFNgamma

### 2.1.2 Reagents

Each cartridge box contained one or more bottles of ready to use Assay Diluent SD13 and Wash Buffer.

### 2.1.3 QCs

1. Spiked Plasma Pool 2017 (stored in 100 μl & 250 μl aliquots at −80° C.)
2. ProteinSimple Low QC (2a=CD14, 2b=CD120b, 2c=IFNg/IL-10/IL-8/CXCL-9, 2d=IP10/IL-12p70/IL-6/TNFa) (stored in 250 μl aliquots at −80° C.)
3. ProteinSimple High QC (3a=CD14, 3b=CD120b, 3c=IFNg/IL-10/IL-8/CXCL-9, 3d=IP10/IL-12p70/IL-6/TNFa) (stored in 250 μl aliquots at −80° C.)

### 2.2 Samples & Batch Format

Samples were sent from various areas of the globe.
NOTE: All samples were regarded as an infection risk and handled with appropriate PPE.
Samples were stored at −20° C. while awaiting analysis and returned to −20° C. storage when analysis was completed.

### 2.2.1 Singleplex Assays

132 samples were thawed for each day's work. These were analysed using 2×CD14 cartridges and 2×CD120b cartridges. Each cartridge had space for 66 unknowns plus 3 QCs at the beginning and 3 QCs at the end. A single 100 μl Spiked Pool aliquot was used each day (or a 250 μl Spiked Pool aliquot each day for the IFNgamma singleplex analysis).

### 2.2.2 Multiplex Assays

56 samples were thawed for each day's work. These were analysed using 2×IFNg 4-plex cartridges and 2×IP10 4-plex cartridges. Each cartridge had space for 28 unknowns plus 2 QCs at the beginning and 2 QCs at the end (spiked pool+Low QC at the beginning, spiked pool+High QC at the end). A single 250 μl Spiked Pool aliquot was used each day.

### 2.3 Assay

### 2.3.1 Singleplex (CD120b & CD14)

Samples & QCs were thawed for a minimum of 15 minutes on a roller-mixer.

Centrifuged samples for 5 minutes at 5000 rpm in an Eppendorf Minifuge.

Centrifuged QCs for 1 minute at 5000 rpm in an Eppendorf Minifuge.

A Singleplex cartridge was sufficient for 66 TB samples plus 3 QCs at the beginning and end of each run.

### Cartridge 1: CD120b (TNF RII)

TB samples & Spiked Pool required a 1 in 10 predilution in Assay Diluent SD13.

For TB samples, 10 μl sample was added to 90 μl Diluent SD13 in a labelled 2 ml Sarstedt tube. Mixed Well.

For Spiked Pool QC, 20 μl sample was added to 180 μl Diluent SD13 in a labelled 2 ml Sarstedt tube. Mixed Well.

### Sample Loading

Pipetted 50 μl into each well
Wells 1 & 2=Low & High QC (undiluted)
Well 3=Spiked Pool QC (diluted)
Wells 4-69=TB samples (diluted)
Well 70=Spiked Pool QC (diluted)
Wells 71 & 72=Low & High QC (undiluted)

Pipetted 1 ml wash buffer into the cartridge wash buffer reservoirs.

When all samples had been loaded the assay was run following the ELLA SOP (see section 3 below).

### Cartridge 2: CD14

Samples & Spiked Pool required a 1 in 5000 predilution in Assay Diluent SD13.

Further diluted the 1 in 10 dilution prepared for the CD120b assay.

10 μl of the 1 in 10 dilution was added to 90 μl Diluent SD13 in a labelled 2 ml Sarstedt tube=1 in 100 dilution. Mixed Well.

5.1 μl of the 1 in 100 dilution was added to 250 μl Diluent SD13 in a labelled 2 ml Sarstedt tube=1 in 5000 dilution. Mixed Well.

### Sample Loading

Pipetted 50 μl into each well
Wells 1 & 2=Low & High QC (undiluted)
Well 3=Spiked Pool QC (diluted)
Wells 4-69=TB samples (diluted)
Well 70=Spiked Pool QC (diluted)
Wells 71 & 72=Low & High QC (undiluted)

Pipetted 1 m1 wash buffer into the cartridge wash buffer reservoirs.

When all samples had been loaded the assay was run following the ELLA SOP (see section 3 below).

When Cartridge 2 was completed the dilution & loading sequences were repeated with a second batch of samples. Cartridge 3=CD120b, Cartridge 4=CD14.

### Cartridge 5: Singleplex IFNgamma

Samples & QCs were thawed for a minimum of 15 minutes on a roller-mixer.
Centrifuged samples for 5 minutes at 5000 rpm in an Eppendorf Minifuge.
Centrifuged QCs for 1 minute at 5000 rpm in an Eppendorf Minifuge.
A Singleplex cartridge was sufficient for 66 TB samples plus 3 QCs at the beginning and end of each run.

### Sample Loading

Pipetted 25 μl Diluent 13 into each well EXCEPT wells 1 & 2 and 71 & 72
Wells 1 & 2=pipette 50 μl Low & High QC (undiluted)
Well 3=pipette 25 μl Spiked Pool QC
Wells 4-69=pipette 25 μl TB samples
Well 70=pipette 25 μl Spiked Pool QC
Wells 71 & 72=pipette 50 μl Low & High QC

Shaken briefly (5 seconds) on a plateshaker to mix.

Pipetted 1 ml wash buffer into the cartridge wash buffer reservoirs.

When all samples had been loaded the assay was run following the ELLA SOP (see section 3 below).

When Cartridge 1 was completed the loading sequence was repeated with a second batch of samples.

2.3.2 Multiplex (IFNgamma & IP10 4-plexes)

Samples & QCs were thawed for a minimum of 15 minutes on a roller-mixer.

Centrifuged samples and QCs for 5 minutes at 5000 rpm in an Eppendorf Minifuge.

Centrifuged QCs for 1 minute at 5000 rpm in an Eppendorf Minifuge.

A Multiplex cartridge was sufficient for 28 TB samples plus 2 QCs at the beginning and end of each run.

### Cartridge 1: IFNgamma+IL-10+IL-8+CXCL-9

TB samples & Spiked Pool required a 1 in 2 dilution in Assay Diluent SD13.

Pipetted 25 μl Assay Diluent SD13 into all of the wells (except for those requiring ProteinSimple QCs) using a Multipette then pipetted 25 μl sample or spiked pools into the wells.

### Sample Loading

Pipetted 25 μl Assay Diluent SD13 into each well EXCEPT wells 1 & 32
Well 1=Low QC 50 μl (undiluted)
Well 2=Spiked Pool QC 25 μl (diluted)
Wells 3-30=TB samples 25 μl (diluted)
Well 31=Spiked Pool QC 25 μl (diluted)
Wells 32=High QC 50 μl (undiluted)

Mixed the plate gently on a plate shaker for 5 seconds.

Pipetted 1 ml wash buffer into the cartridge wash buffer reservoirs.

When all samples & wash buffers had been loaded the assay was run following the ELLA SOP (see section 3 below).

### Cartridge 2: IP10+IL-12p70+IL-6+TNFalpha

TB samples & Spiked Pool required a 1 in 2 dilution in Assay Diluent SD13. Pipetted 25 μl Assay Diluent SD13 into all of the wells (except for those requiring ProteinSimple QCs) using a Multipette then pipetted 25 μl sample or spiked pools into the wells.

### Sample Loading

Pipetted 25 μl Assay Diluent SD13 into each well EXCEPT wells 1 & 32
Well 1=Low QC 50 μl (undiluted)
Well 2=Spiked Pool QC 25 μl (diluted)
Wells 3-30=TB samples 25 μl (diluted)
Well 31=Spiked Pool QC 25 μl (diluted)
Wells 32=High QC 50 μl (undiluted)

Mixed the plate gently on a plate shaker for 5 seconds.

Pipetted 1 ml wash buffer into the cartridge wash buffer reservoirs.

When all samples & wash buffers had been loaded the assay was run following the ELLA SOP (see section 3 below).

When Cartridge 2 was completed the loading sequences were repeated with a second batch of samples. Cartridge 3=IFNg 4-plex, Cartridge 4=IP10 4-plex.

### 3. Operation of the ProteinSimple ELLA Immunoassay Analyser

### 3.1 Instrument Manufacturer

The instrument is manufactured by ProteinSimple (a BioTechne subsiduary).

### 3.2 Assay Description

The ELLA instrument is capable of performing singleplex or multiplex immunoassays with minimal user hands-on time. Assays were performed in a solid microtitre plate sized cartridge which contained a network of microfluidic channels. The cartridges were configured to measure 72 samples for a single biomarker or 16 or 32 samples for up to 4 biomarkers.

The ELLA cartridges are factory-calibrated. Samples (pre-diluted if required), QC and wash buffers were loaded into the cartridge by the operator. The instrument was programmed and the cartridge loaded. The whole immunoassay procedure is fully automated. Results were available in approximately 90 minutes. Each sample was loaded in singleton but measurements were performed in triplicate in ‘glass nano reactors’ (GNR).

The assay was run in accordance with the manufacturers' instructions.

### Results

### Example 1—Performance of Predictive Models in Different Diagnostic Groups—IDEA Cohort

The percentage of correctly diagnosed patients in each group (as stratified above) was calculated from predictive models generated using both the 5BM and 7BM biomarker panels. Table 4 shows the predictive performance for each of these models and biomarker panels. As can be seen, predictive models generated using both biomarker panels are able to correctly diagnose active TB.

TABLE 4
Performance of predictive models in different
diagnostic groups-IDEA cohort:

| 570 training samples | | | HIV −ve | | HIV +ve | |
|---|---|---|---|---|---|---|
| 246 test samples | | | 5BM | 7BM | 5BM | 7BM |
| 1 | active TB | smear +ve | 88% | 92% | 100% | 100% |
| | | | (65/74) | (68/74) | (5/5) | (5/5) |
| 2 | | smear −ve | 92% | 96% | 25% | 25% |
| | | | (24/26) | (25/26) | (1/4) | (1/4) |
| 4A | latent | exposure +ve | 73% | 80% | 100% | 88% |
| | TST +ve | | (11/15) | (12/15) | (8/8) | (7/8) |
| 4B | | exposure −ve | 82% | 82% | n/a | n/a |
| | | | (9/11) | (9/11) | (0/0) | (0/0) |
| 4C | nonTB | exposure +ve | 67% | 64% | 67% | 67% |
| | TST −ve | | (37/55) | (35/55) | (14/21) | (14/21) |
| 4D | | exposure −ve | 72% | 67% | 78% | 78% |
| | | | (10/18) | (12/18) | (7/9) | (7/9) |

The fraction of correctly diagnosed patients is shown in brackets.

### Example 2—Performance of Predictive Models in Different Diagnostic Groups—SA Cohort

The percentage of correctly diagnosed patients in each group (see stratification above) of a cohort independent from the IDEA cohort as presented in Example 1 was calculated from predictive models generated using both the 5BM and 7BM biomarker panels. Table 5 shows the predictive performance for each of these models and biomarker panels. As can be seen, predictive models generated using both biomarker panels are able to correctly diagnose active TB.

TABLE 5
Performance of predictive models in different
diagnostic groups-SA cohort:

| | | | HIV −ve | | HIV +ve | |
|---|---|---|---|---|---|---|
| 179 test samples | | | 5BM | 7BM | 5BM | 7BM |
| SA1 | active TB | definite | 93% | 91% | 98% | 98% |
| | | | (41/44) | (40/44) | (42/43) | (42/43) |
| SA2 | | highly | n/a | n/a | 67% | 100% |
| | | probable | (0/0) | (0/0) | (2/3) | (3/3) |
| SA3 | non-TB | active | 83% | 83% | 89% | 83% |
| | | excluded | (43/52) | (43/52) | (16/18) | (15/18) |
| SA4 | | healthy | 95% | 89% | n/a | n/a |
| | | | (18/19) | (17/19) | (0/0) | (0/0) |

The fraction of correctly diagnosed patients is shown in brackets.

### Example 3—Performance of Predictive Models Using ELLA Device

In order to further validate the benefits of the 5BM and 7BM biomarker panels, a further evaluation was conducted on a separate, next generation, ELISA device known as ELLA (manufactured by ProteinSimple). ELLA utilizes fluorescence chemistry which differs from the evaluations described hereinbefore which utilize electrochemiluminescence.

The present test was validated with a total of 1376 samples supplied from tuberculosis clinics in 4 countries (Spain, UK, South Africa and Brazil) of people presenting with tuberculosis symptoms, plus controls.

The present test was performed on the ELLA device following the protocols provided hereinbefore.

The results for the 5BM and 7BM biomarker panels are shown below and pictorially in FIGS. 6 and 7, respectively:

TABLE 6
Performance of predictive models on ELLA device

| Biomarker | | | | Area Under Curve |
|---|---|---|---|---|
| Panel | Sensitivity | Specificity | Accuracy | (AUC) |
| 5BM | 0.92 | 0.7 | 0.77 | 0.89 |
| 7BM | 0.91 | 0.76 | 0.80 | 0.91 |

The predictive models were trained on the datasets from the earlier studies as described hereinbefore. The data from the ELLA assay were transformed using a robust linear regression transformation. The data presented in Table 6 and FIGS. 6 and 7, show that both predictive models (5BM and 7BM) performed well and are consistent with previously generated data. Noteworthy, the ELLA device utilizes an entirely different detection chemistry.

1. Use of a panel comprising the biomarkers IFN gamma, IL-10, CD120b and CD14 for diagnosing, and/or monitoring the progression of, an active mycobacterial infection.

2. The use according to claim 1, wherein the panel additionally comprises CXCL10 (IP-10).

3. The use according to claim 1, wherein the panel additionally comprises at least one of IL-8, IL-12p70 and CXCL9.

4. The use according to claim 1, wherein the panel additionally comprises at least two of IL-8, IL-12p70 and CXCL9.

5. The use according to claim 1, wherein the panel additionally comprises IL-8, IL-12p70 and CXCL9.

6. The use according to any one of claims 1 to 5, wherein the panel further comprises the HIV status of an individual.

7. A method of diagnosing the absence of an active mycobacterial infection in an individual, comprising:
(a) obtaining a test biological sample from an individual;
(b) quantifying the amount of the biomarkers as defined in any one of claims 1 to 6;
(c) inputting said amounts into a prediction model generated by an algorithm which comprises the relative amounts of each biomarker in active mycobacterial infected patients;
(d) obtaining a likelihood value for the absence of an active mycobacterial infection from said model.

8. A method of excluding the presence, and/or monitoring the progression of, an active mycobacterial infection in an individual thereto, comprising:
(a) obtaining a test biological sample from an individual;
(b) quantifying the amount of the biomarkers as defined in any one of claims 1 to 6;
(c) comparing the amounts of one or more of the biomarkers in the test biological sample with the amounts present in one or more control samples, such that a difference in the level of one or more of the biomarkers in the test biological sample is indicative of the absence of an active mycobacterial infection.

9. A method of determining the efficacy of anti-mycobacterial therapy for an active mycobacterial infection in an individual subject comprising:
(a) obtaining a biological sample from an individual;
(b) quantifying the amount of the biomarkers as defined in any one of claims 1 to 6;
(c) comparing the amounts of one or more of the biomarkers in the test biological sample with the amounts present in one or more control samples, such that a difference in the level of one or more of the biomarkers in the test biological sample is indicative of a response to the treatment.

10. The use according to any one of claims 1 to 6 or the method according to any one of claims 7 to 9, wherein the active mycobacterial infection is active tuberculosis.

11. The use according to any one of claims 1 to 6 or the method according to any one of claims 7 to 9, wherein the active mycobacterial infection is extrapulmonary tuberculosis.

12. The use according to any one of claims 1 to 6 or the method according to any one of claims 7 to 9, wherein the active mycobacterial infection is not active tuberculosis or extrapulmonary tuberculosis.

13. The use according to any one of claims 1 to 6 or the method according to any one of claims 7 to 8 and 10 to 11, wherein the diagnosis or excluding the presence of a mycobacterial infection is differential diagnosis between:
(i) active tuberculosis patients and patients with latent, non-progressing tuberculosis or sick patients or healthy patients; and/or
(ii) the patient groups of (i), irrespective of whether they have been characterised as being sputum smear positive or sputum smear negative; and/or
(iii) the patient groups of (i) and/or (ii), irrespective of whether they have been characterised as being HIV positive or HIV negative.

14. The method as defined in any one of claims 7 to 13, which is conducted on samples taken on two or more occasions from a test subject.

15. The method as defined in claim 14, further comprising detecting a change in the amount of the biomarkers in samples taken on two or more occasions.

16. The method as defined in any one of claims 7 to 15, wherein the quantifying is performed by measuring the concentration of the analyte biomarkers in each sample.

17. The method as defined in any of one of claims 7 to 16, wherein the detecting and/or quantifying is performed using an immunological method.

18. The method as defined in any one of claims 7 to 17, wherein the detecting and/or quantifying is performed using a biosensor or a microanalytical, microengineered, microseparation or immunochromatography system.

19. The method as defined in any one of claims 7 to 18, wherein the biological sample is whole blood, serum, plasma, tissue fluid, cerebrospinal fluid (CSF), synovial fluid, follicular fluid, seminal fluid, amniotic fluid, milk, urine, pleural fluid, ascites, bronchoalveolar lavage, saliva, sputum, tears, perspiration, lymphatic fluid, aspirate, bone marrow aspirate and mucus, or an extract or purification therefrom, or dilution thereof.

20. The method as defined in claim 19, wherein the biological sample is whole blood, serum or plasma.

21. The method as defined in claim 19 or claim 20, wherein the biological sample is serum, such as non-activated serum or serum activated using disease specific antigens, such as Mycobacterium tuberculosis specific antigens.

22. The method as defined in claim 21, wherein the method of excluding the presence, monitoring the progression or diagnosing the absence of an active mycobacterial infection, active tuberculosis or extrapulmonary tuberculosis comprises:
(a) obtaining a serum sample from an individual;
(b) dividing said serum sample into a serum sample for activation using disease specific antigens, such as Mycobacterium tuberculosis specific antigens, and a non-activated serum sample;
(c) activating said serum sample using disease specific antigens, such as Mycobacterium tuberculosis specific antigens;
(d) quantifying the amount of the biomarkers as defined in any one of claims 1 to 6 in each of the activated and non-activated serum samples;
(e) comparing the amounts of one or more of the biomarkers in the activated serum sample with the amounts present in the non-activated serum sample, such that a difference in the level of one or more of the biomarkers in the activated serum sample is indicative of:
(i) a diagnosis of tuberculosis; and/or
(ii) the presence of tuberculosis infection rather than any other mycobacterial infection.

23. A method of treating an active mycobacterial infection in an individual in need thereof, wherein said method comprises the following steps:
(a) excluding the presence, and/or monitoring the progression of, an active mycobacterial infection in an individual according to the method as defined in any one of claims 7 to 8 and 10 to 22; followed by
(b) administering an anti-mycobacterial medicament to said individual in the event of a positive diagnosis for active mycobacterial infection.

24. The method of treating an active mycobacterial infection as defined in claim 23, wherein the active mycobacterial infection is active tuberculosis or extrapulmonary tuberculosis and wherein the anti-mycobacterial medicament is an anti-tuberculosis medicament.

25. The method as defined in claim 23 or claim 24, wherein said anti-mycobacterial or anti-tuberculosis medicament is: one or more first line medicaments selected from: ethambutol, isoniazid, pyrazinamide, rifampicin; and/or one or more second line medicaments selected from: aminoglycosides (e.g., amikacin, kanamycin), polypeptides (e.g., capreomycin, viomycin, enviomycin), fluoroquinolones (e.g., ciprofloxacin, levofloxacin, moxifloxacin), thioamides (e.g. ethionamide, prothionamide), cycloserine (closerin) or terizidone; and/or one or more third line medicaments selected from rifabutin, macrolides (e.g., clarithromycin), linezolid, thioacetazone, thioridazine, arginine, vitamin D and R207910.

26. A kit comprising a biosensor capable of detecting and/or quantifying the amount of the biomarkers as defined in any one of claims 1 to 6, for use in diagnosing, and/or monitoring the progression of, an active mycobacterial infection.

27. The kit as defined in claim 26, wherein the active mycobacterial infection is active tuberculosis.

28. The kit as defined in claim 26 or claim 27, wherein the active mycobacterial infection or active tuberculosis is extrapulmonary tuberculosis.

29. The kit as defined in claim 26, wherein the active mycobacterial infection is not active tuberculosis or extrapulmonary tuberculosis.
